(19) United States
(12) Patent Application Publication
(10) Pub. No.: US 2023/0357418 A1
(43) Pub. Date: Nov. 9, 2023

(54) RESULTS OF EMPACTA: A RANDOMIZED, DOUBLE-BLIND, PLACEBO-CONTROLLED, MULTICENTER STUDY TO EVALUATE THE EFFICACY AND SAFETY OF TOCILIZUMAB IN HOSPITALIZED PATIENTS WITH COVID-19 PNEUMONIA

(71) Applicant: Genentech, Inc., South San Francisco, CA (US)

(72) Inventors: Shalini V. MOHAN, San Francisco, CA (US); William George REISS, Lititz, PA (US); Hoi Ying Linda YAU, Sunnyvale, CA (US); Benjamin Howard KRAMER, San Francisco, CA (US); Jian HAN, Redwood City, CA (US)

(73) Assignee: Genentech, Inc., South San Francisco, CA (US)

(21) Appl. No.: 18/245,372

(22) PCT Filed: Mar. 19, 2021
(86) PCT No.: PCT/US2021/023099

§ 371 Date: Mar. 15, 2023

Related U.S. Application Data
(60) Provisional application No. 63/079,877, filed on Sep. 17, 2020.

(51) Int. Cl. C07K 16/28; A61K 31/706; A61K 31/573; A61P 11/00

(52) U.S. Cl. CPC C07K 16/2866; A61K 31/706; A61K 31/573; A61P 11/00; A61K 2039/545

(57) ABSTRACT

The application disclosed concerns a method of treating pneumonia in a patient who is not mechanically ventilated comprising administering an IL-6 antagonist (e.g. tocilizumab) to the patient in an amount effective to prevent the patient from dying or receiving mechanical ventilation. The disclosure is based on the results of the EM PACTA clinical trial.

### CROSS REFERENCE TO RELATED APPLICATION

This application claims priority to U.S. Provisional Application No. 63/079,877 filed on Sep. 17, 2020, the disclosure of which is incorporated herein by reference in its entirety.

### SEQUENCE LISTING

The instant application contains a sequence listing submitted via efs-web and is hereby incorporated by reference in its entirety. Said ASCII copy, created Mar. 15, 2021, is named P36402WOSEQLIST.txt, and is 7,434 bytes in size.

### FIELD OF THE INVENTION

The invention concerns methods of treating pneumonia in patients with an IL-6 antagonist. It includes methods for treating viral pneumonia, such as coronavirus pneumonia, and exemplified by COVID-19 pneumonia. In particular, the invention disclosed concerns a method of treating pneumonia in a patient who is not mechanically ventilated comprising administering an IL-6 antagonist (e.g. tocilizumab) to the patient in an amount effective to prevent the patient from dying or receiving mechanical ventilation.

### BACKGROUND OF THE INVENTION

Interleukin-6 (IL-6) is a proinflammatory, multifunctional cytokine produced by a variety of cell types. IL-6 is involved in such diverse processes as T-cell activation, B-cell differentiation, induction of acute phase proteins, stimulation of hematopoietic precursor cell growth and differentiation, promotion of osteoclast differentiation from precursor cells, proliferation of hepatic, dermal and neural cells, bone metabolism, and lipid metabolism (Hirano T. Chem Immunol. 51:153-180 (1992); Keller et al. Frontiers Biosci. 1: 340-357 (1996); Metzger et al. Am J Physiol Endocrinol Metab. 281: E597-E965 (2001); Tamura et al. Proc Natl Acad Sci USA. 90:11924-11928 (1993); Taub R. J Clin Invest 112: 978-980 (2003)). IL-6 has been implicated in the pathogenesis of a variety of diseases including autoimmune diseases, osteoporosis, neoplasia, and aging (Hirano, T. (1992), supra; and Keller et al., supra). IL-6 exerts its effects through a ligand-specific receptor (IL-6R) present both in soluble and membrane-expressed forms.

Elevated IL-6 levels have been reported in the serum and synovial fluid of rheumatoid arthritis (RA) patients, indicative of production of IL-6 by the synovium (Irano et al. Eur J Immunol. 18:1797-1801 (1988); and Houssiau et al. Arthritis Rheum. 1988; 31:784-788 (1988)). IL-6 levels correlate with disease activity in RA (Hirano et al. (1988), supra), and clinical efficacy is accompanied by a reduction in serum IL-6 levels (Madhok et al. Arthritis Rheum. 33:S154. Abstract (1990)).

Tocilizumab (TCZ) is a recombinant humanized monoclonal antibody of the immunoglobulin IgG1 subclass which binds to human IL-6 receptor. Clinical efficacy and safety studies of intravenous (iv) TCZ have been completed or are conducted by Roche and Chugai in various disease areas, including adult-onset RA, systemic juvenile idiopathic arthritis (sJIA) and polyarticular juvenile idiopathic arthritis (pJIA).

Tocilizumab is approved in the United States for:
1. Rheumatoid Arthritis (RA): Adult patients with moderately to severely active rheumatoid arthritis who have had an inadequate response to one or more Disease-Modifying Anti-Rheumatic Drugs (DMARDs).
2. Giant Cell Arteritis (GCA): Adult patients with giant cell arteritis.
3. Polyarticular Juvenile Idiopathic Arthritis (pJIA): Patients 2 years of age and older with active polyarticular juvenile idiopathic arthritis.
4. Systemic Juvenile Idiopathic Arthritis (sJIA): Patients 2 years of age and older with active systemic juvenile idiopathic arthritis.
5. Cytokine Release Syndrome (CRS): Adults and pediatric patients 2 years of age and older with chimeric antigen receptor (CAR) T cell-induced severe or life-threatening cytokine release syndrome.

Coronaviruses (CoV) are positive-stranded RNA viruses with a crown-like appearance under an electron microscope due to the presence of spike glycoproteins on the envelope. They are a large family of viruses that cause illness ranging from the common cold to more severe diseases such as Middle East respiratory syndrome (MERS-CoV) and severe acute respiratory syndrome (SARS-CoV).

COVID-19, which is the acronym of “coronavirus disease 2019,” is caused by a new coronavirus strain that has not been previously identified in humans and was newly named on 11 Feb. 2020 by the World Health Organization (WHO). An epidemic of cases with unexplained lower respiratory tract infections was first detected in Wuhan, the largest metropolitan area in China's Hubei province, and was reported to the WHO Country Office in China on Dec. 31, 2019. A pandemic was subsequently declared by the WHO on 11 Mar. 2020.

According to the WHO, as of 17 Mar. 2020 over 179,000 cases of COVID-19 were reported in over 100 countries worldwide, with over 7400 deaths. Up to ˜20% of infected patients experienced complications related to a severe form of interstitial pneumonia, which may progress towards acute respiratory distress syndrome (ARDS) and/or multi organ failure (MOF) and death.

CRS has been identified as a clinically significant, on-target, off-tumor side effect of the CAR T-cell therapies used for treatment of malignancies. Characteristics of CRS include fever, fatigue, headache, encephalopathy, hypotension, tachycardia, coagulopathy, nausea, capillary leak, and multi-organ dysfunction. The reported incidence of CRS after CAR T-cell therapy ranges from 50% to 100%, with 13% to 48% of patients experiencing the severe or life-threatening form. Serum levels of inflammatory cytokines are elevated, particularly interleukin-6 (IL-6). The severity of symptoms may correlate with the serum cytokine concentrations and the duration of exposure to the inflammatory cytokines.

On Aug. 30, 2017, the U.S. Food and Drug Administration approved tocilizumab (ACTEMRA®) for the treatment of severe or life-threatening CAR T cell-induced CRS in adults and in pediatric patients 2 years of age and older. The approved dose is 8 mg/kg for body weight ≥30 kg and 12 mg/kg for body weight <30 kg. Up to three additional doses may be given if no improvement of sign/symptoms, and the interval between the subsequent doses should be at least 8 hours.

The approval of TCZ was based on a retrospective analysis of data for patients treated with TCZ who developed CRS after treatment with tisagenlecleucel (KYMRIAH®) or axicabtagene ciloleucel (YESCARTA®) in prospective clinical trials (Le et al. The Oncologist. 23:943-947 (2018)). Thirty-one out of the 45 patients (69%) from the CTL019 series achieved a response (defined as being afebrile and off vasopressors for at least 24 hours within 14 days of the first dose of TCZ (maximum up to two doses) and without use of additional treatment other than corticosteroids) within 14 days of the first dose of TCZ, and the median time from the first dose to response was 4 days. Eight of the 15 patients (53%) from the axicabtagene ciloleucel series achieved a response, and the median time to response was 4.5 days. The response rates were largely consistent among subgroups such as age group, sex, race, ethnicity, grade of CRS at first dose of TCZ, and duration of CRS prior to treatment with TCZ. There were no reports of adverse reactions attributable to TCZ.

Pharmacokinetic (PK) data were available for 27 patients after the first dose of TCZ and for 8 patients after a second dose of TCZ. Based on 131 PK observations, the geometric mean (% CV) maximum concentration of TCZ in the patients with CAR T cell induced, severe or life-threatening CRS was 99.5 μg/mL (36.8%) after the first infusion and 160.7 μg/mL (113.8%) after the second infusion. The PK modeling analysis showed that patients with CRS had a faster clearance of TCZ than healthy volunteers and other patient populations, and simulations showed that exposure was considered acceptable with up to four doses of TCZ at least 8 hours apart in patients with CRS.

TCZ is also approved for CAR-T induced severe or life-threatening CRA in European Union and certain other countries.

Physicians in China initiated the off-label usage of TCZ in the treatment of coronavirus (COVID-19) pneumonia. Based on the findings of an observational study of 21 COVID-19 patients treated with TCZ, an investigator-initiated randomized, open-label study (n=188) was also initiated on 13 Feb. 2020.

On 3 Mar. 2020, TCZ was included in the Seventh Edition “Diagnosis and Treatment Protocol of COVID-19 Pneumonia” by the China National Health Commission as one treatment option for severe or critical forms of COVID-19 pneumonia. The Chinese CDC defined disease severity according to the following criteria:
1. Severe pneumonia: dyspnea, respiratory frequency 30/min. blood oxygen saturation (SpO₂)≤93%, PaO2/FiO₂ ratio [the ratio between the blood pressure of the oxygen (partial pressure of oxygen, PaO2) and the percentage of oxygen supplied (fraction of inspired oxygen, FiO₂)]<300 mmHg, and/or lung infiltrates >50% within 24 to 48 hours; this occurred in 14% of cases.
2. Critical pneumonia: respiratory failure, septic shock, and/or multiple organ dysfunction (MOD) or failure (MOF): this occurred in 5% of cases (Wu et al. JAMA. doi:10.1001/jama.2020.2648 (2020)).

According to Section 10.3.7 of these Guidelines: “For patients with extensive lung lesions and severe patients, and laboratory testing of elevated IL-6 levels, tocilizumab treatment can be tried. The first dose is 4 to 8 mg/kg, the recommended dose is 400 mg, 0.9% saline is diluted to 100 ml, and the infusion time is more than 1 hour; if no clinical improvement in the signs and symptoms occurs after the first dose, it can be applied at the same dose as before more after 12 hours. The cumulative number of administrations is a maximum of 2 times, and the maximum single dose does not exceed 800 mg. Pay attention to hypersensitivity, and those with active infection such as tuberculosis are contraindicated.”

Based on the results of an initial 21-patient retrospective observational study in which patients with severe or critical coronavirus (COVID-19) pneumonia were treated with TCZ, a randomized, controlled trial (n=188) has been initiated in the same population testing the same TCZ dose regimen and is currently ongoing with approximately 70 patients enrolled. Xu et al. Effective treatment of severe COVID-19 patients with tocilizumab. Submitted manuscript. [Resource on the internet]. 2020 [updated 5 Mar. 2020; cited 17 Mar. 2020]. Available from: http://www.chinaxiv.org/abs/202003.00026.

In February 2020, twenty-one patients with severe or critical COVID-19 pneumonia were treated with TCZ IV (400 mg) plus standard of care. The average age of the patients was 56.8±16.5 years, ranging from 25 to 88 years. Seventeen patients (81.0%) were assessed as severe and four (19.0%) as critical. Most patients (85%) presented with lymphopenia. C-reactive protein (CRP) levels were increased in all 20 patients (mean, 75.06±66.80 mg/L). The median procalcitonin (PCT) value was 0.33±0.78 ng/mL, and only two of 20 patients (10.0%) presented with an abnormal value. Mean IL-6 level before TCZ was 132.38±278.54 pg/mL (normal <7 pg/mL).

Standard of care consisted of lopinavir, methylprednisolone, other symptom relievers. and oxygen therapy as recommended by the Diagnosis and Treatment Protocol for Novel Coronavirus Pneumonia (Sixth Edition). All 21 patients had received routine standard of care treatment for a week before deteriorating with sustained fever, hypoxemia, and chest CT image worsening.

Eighteen patients (85.7%) received TCZ once, and three patients (14.3%) had a second dose due to fever within 12 hours. According to the authors, after TCZ treatment. fever returned to normal and all other symptoms improved remarkably. Fifteen of the 20 patients (75.0%) had lowered their oxygen intake and one patient needed no oxygen therapy. CT scans showed significant remission of opacities in both lungs in 19/20 patients (90.5%) after treatment with TCZ. The percentage of lymphocytes in peripheral blood, which was decreased in 85.0% of patients (17/20) before treatment (mean, 15.52±8.89%), returned to normal in 52.6% of patients (10/19) on the fifth day after treatment. Abnormally elevated CRP decreased significantly in 84.2% patients (16/19). No adverse drug reactions and no subsequent pulmonary infections were reported.

Nineteen patients (90.5%) were discharged at the time of the report, including two critical patients. There were no deaths among the 21 treated patients. The study authors concluded that TCZ is an effective treatment for patients with severe COVID-19 (Xu et al (2020), supra).

Clinical trials related to tocilizumab for COVID-19 pneumonia include, inter alia:
1. A Study to Evaluate the Safety and Efficacy of Tocilizumab in Patients With Severe COVID-19 Pneumonia (COVACTA): ClinicalTrials.gov Identifier NCT04320615, first posted: Mar. 25, 2020. The initial results of COVACTA were announced in the following Press Release: https://www.roche.com/investors/updates/inv-update-2020-07-29.htm. COVACTA trial did not meet its primary endpoint of improved clinical status in patients with COVID-19 associated pneumonia, or the key secondary endpoint of reduced patient mortality. Time to hospital discharge or ‘ready to discharge’ was shorter in patients treated with Actemra/RoActemra than in those treated with placebo. The median time to discharge or ‘ready to discharge’ for Actemra/RoActemra was 20 days and for placebo was 28 days (median time 195% CII: Actemra/RoActemra=20.0 [17.0, 27.0]; placebo=28.0 [20.0. NE], p=0.0370). The difference in ventilator-free days between Actemra/RoActemra and placebo was not statistically significant (median of 22 days for Actemra/RoActemra and 16.5 days with placebo, difference in medians [95% CI]=5.5 [−2.8, 13.0], p=0.3202).
2. A Study to Evaluate the Efficacy and Safety of Remdesivir Plus Tocilizumab Compared With Remdesivir Plus Placebo in Hospitalized Participants With Severe COVID-19 Pneumonia (REWDACTA): ClinicalTrials.gov Identifier NCT04409262, first posted: Jun. 1, 2020.
3. A Study to Evaluate the Efficacy and Safety of Tocilizumab in Hospitalized Participants With COVID-19 Pneumonia (EMPACTA): ClinicalTrials.gov Identifier NCT04372186, first posted: May 1, 2020.
4. A Study to Investigate Intravenous Tocilizumab in Participants With Moderate to Severe COVID-19 Pneumonia (MARIPOSA): ClinicalTrials.gov Identifier NCT04363736, first posted: Apr. 27, 2020.
5. Tocilizumab to Prevent the Progression of Hypoxemic Respiratory Failure in Hospitalized Non-Critically Ill Patients With COVID-19 (MGH Study): ClinicalTrials.gov Identifier: NCT04356937, first posted: Apr. 22, 2020. This study includes as “Inclusion Criteria” at least 1 of the following: a. Ferritin >500 ng/ml (which is >1124 pmol/L), CRP>50 mg/L, c. LDH>250 U/L, d. D-dimer >1000 ng/mL.

An adaptive Phase 2/3, randomized, double-blind, placebo-controlled study assessing efficacy and safety of Sarilumab for hospitalized patients with COVID-19 is found at: ClinicalTrials.gov Identifier: NCT04315298, first posted: Mar. 19, 2020. Sarilumab is a human monoclonal antibody against the interleukin-6 receptor.

### SUMMARY OF THE INVENTION

In a first aspect, the invention concerns a method of treating pneumonia in a patient who is not mechanically ventilated comprising administering an IL-6 antagonist to the patient in an amount effective to prevent the patient from dying or receiving mechanical ventilation.

In a second aspect, the invention concerns a method of treating viral pneumonia in a patient comprising who is not mechanically ventilated comprising administering a combination of an IL-6 antagonist and remdesivir to the patient in an amount effective to prevent the patient from dying or receiving mechanical ventilation.

In another aspect, the invention concerns a method of treating viral pneumonia in a patient who is not mechanically ventilated comprising administering a combination of an IL-6 antagonist and a corticosteroid to the patient in an amount effective to prevent the patient from dying or receiving mechanical ventilation.

### BRIEF DESCRIPTION OF THE DRAWINGS

FIG. 1 depicts EMPACTA study design.

FIG. 2 depicts patient disposition.

FIGS. 3A-B depict patient demographics.

FIGS. 4A-C depict baseline disease characteristics.

FIG. 5 depicts study drug exposure (safety population).

FIG. 6 depicts primary efficacy endpoint: cumulative proportion of patients with death or requiring mechanical ventilation by Day 28.

FIGS. 7-10 depict secondary efficacy endpoints, namely:
FIG. 7: time to hospital discharge or “ready for discharge” up to Day 28.
FIG. 8: time to improvement in ordinal clinical status up to Day 28.
FIG. 9: time to clinical failure up to Day 28.
FIG. 10: mortality by Day 28.

FIG. 11 depicts safety overview.

FIG. 12 depicts adverse events of special interest.

FIG. 13 depicts serious adverse events by system organ class (incidence >1% in any group).

FIG. 14 depicts serious adverse events: infections and infestations (incidence >1% in any group).

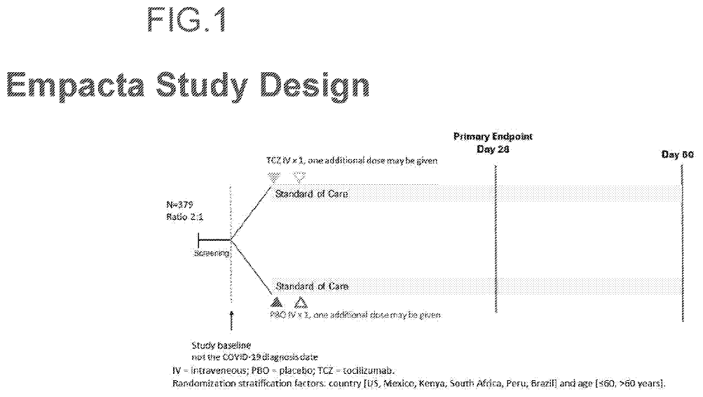

### DETAILED DESCRIPTION OF THE PREFERRED EMBODIMENTS

### I. Definitions

Abbreviations that may be used in this description:

| Abbreviation | Definition |
|---|---|
| ALT | alanine aminotransferase |
| ANC | absolute neutrophil count |
| ARDS | acute respiratory distress syndrome |
| AST | aspartate transaminase |
| AUC | area under the curve |
| BAL | bronchoalveolar lavage |
| CAR | chimeric antigen receptor |
| Cₘₐₓ | maximum serum concentration observed |
| CMH | Cochran-Mantel-Haenszel |
| CoV | Coronaviruses |
| CRP | C-reactive protein |
| CRS | cytokine-release syndrome |
| CTCAE | Common Terminology Criteria for Adverse Events |
| ECMO | extracorporeal membrane oxygenation |
| FDA | Food and Drug Administration |
| GCA | giant cell arteritis |
| ICU | intensive care unit |
| IL-6 | interleukin 6 |
| IL-6R | interleukin-6 receptor |
| Iv | intravenous |
| MERS-CoV | Middle East respiratory syndrome |
| MOD | multiple organ dysfunction |
| MOF | multi organ failure |
| NEWS2 | National Early Warning Score 2 |
| PaO₂ | partial pressure of oxygen |
| PCR | polymerase chain reaction |
| pJIA | polyarticular juvenile idiopathic arthritis |
| PK | Pharmacokinetic |
| QW | once a week |
| Q2W | every 2 weeks |
| RA | rheumatoid arthritis |
| RT-PCR | real time polymerase chain reaction |
| SAE | serious adverse event |
| SAP | Statistical Analysis Plan |
| SARS | severe acute respiratory syndrome |
| Sc | subcutaneous |
| sIL-6-R | soluble interleukin-6 receptor |
| sJIA | systemic juvenile idiopathic arthritis |
| SOC | standard of care |
| SpO₂ | blood oxygen saturation |
| TAK | Takayasu arteritis |
| TB | Tuberculosis |
| TCZ | Tocilizumab |
| TTCI | time to clinical improvement |
| ULN | upper limit of normal |
| WHO | World Health Organization |

For the purposes herein “inflammation” refers to an immunological defense against infection, marked by increases in regional blood flow, immigration of white blood cells, and release of chemical toxins. Inflammation is one way the body uses to protect itself from infection. Clinical hallmarks of inflammation include redness, heat, swelling, pain, and loss of function of a body part. Systemically, inflammation may produce fevers, joint and muscle pains, organ dysfunction, and malaise.

“Pneumonia” refers to inflammation of one or both lungs, with dense areas of lung inflammation. The present invention concerns pneumonia due to viral infection. Symptoms of pneumonia may include fever, chills, cough with sputum production, chest pain, and shortness of breath. In one embodiment the pneumonia has been confirmed by chest X-ray or computed tomography (CT scan).

“Severe pneumonia” refers to pneumonia in which the heart, kidneys or circulatory system are at risk of failing, or if the lungs can no longer take in sufficient oxygen and develop acute respiratory distress syndrome (ARDS). A patient with severe pneumonia will typically be hospitalized and may be in an intensive care unit (ICU). Typically, the patient has severe dyspnea, respiratory distress, tachypnea (>30 breaths/min), and hypoxia, optionally with fever. Cyanosis can occur in children. In this definition, the diagnosis is clinical, and radiologic imaging is used for excluding complications. In one embodiment, the patient with severe pneumonia has impaired lung function as determined by peripheral capillary oxygen saturation (SpO₂). In one embodiment, the patient with severe pneumonia has impaired lung function as determined by ratio of arterial oxygen partial pressure to fractional inspired oxygen (PaO2/FiO₂). In one embodiment, the patient with severe pneumonia has a SpO₂≤93%. In one embodiment, the patient with severe pneumonia has a PaO2/FiO_{2 of} <300 mmHg (optionally adjusted for high altitude areas based on PaO2/FiO₂×[Atmospheric Pressure (mmHg)/760]). In one embodiment, the patient has respiratory distress (RR >30 breaths/minute). In one embodiment, the patient has >50% lesions in pulmonary imaging.

“Critical pneumonia” refers to a severe pneumonia patient in whom respiratory failure, shock and/or organ has occurred. In one embodiment, the patient with critical pneumonia requires mechanical ventilation.

“Mild pneumonia” presents with symptoms of an upper respiratory tract viral infection, including mild fever, cough (dry), sore throat, nasal congestion, malaise, headache, muscle pain, or malaise. Signs and symptoms of a more serious disease, such as dyspnea, are not present.

In “moderate pneumonia”, respiratory symptoms such as cough and shortness of breath (or tachypnea in children) are present without signs of severe pneumonia. The patient with moderate pneumonia may be in a hospital, but not in an ICU or on a ventilator.

“Acute respiratory disease syndrome” or “ARDS” refers to a life-threatening lung condition that prevents enough oxygen from getting to the lungs and into the blood. In one embodiment, the diagnosis of ARDS is made based on the following criteria: acute onset, bilateral lung infiltrates on chest radiography of a non-cardiac origin, and a PaO/FiO ratio of <300 mmHg. In one embodiment, the ARDS is “mild ARDS” characterized by PaO2/FiO2 200 to 300 mmHg. In one embodiment, the ARDS is “moderate ARDS” characterized by PaO2/FiO2 100 to 200 mmHg. In one embodiment, the ARDS is “severe ARDS” characterized by PaO2/FiO2<100 mmHg.

“Viral pneumonia” refers to pneumonia caused by the entrance into a patient of one or more viruses. In one embodiment, the virus is a DNA virus. In one embodiment, the virus is an RNA virus. Examples of viruses causing viral pneumonia contemplated herein include, inter alia, those caused by: human immunodeficiency virus (HIV), hepatitis B virus, hepatitis C virus, influenza virus (including H1N1 or “swine flu” and H5N1 or “bird flu”), Zika virus, rotavirus, Rabies virus, West Nile virus, herpes virus, adenovirus. respiratory syncytial virus (RSV), norovirus, rotavirus, astrovirus, rhinovirus, human papillomavirus (HPV), polio virus, Dengue fever, Ebola virus, and coronavirus. In one embodiment, the viral pneumonia is caused by a coronavirus.

“Coronavirus” is a virus that infects humans and causes respiratory infection. Coronaviruses that can cause pneumonia in patients include, without limitation, the beta coronavirus causes Middle East Respiratory Syndrome (MERS), the beta coronavirus that causes severe acute respiratory syndrome (SARS), and the SARS-CoV-2 virus that causes COVID-19.

“COVID-19” refers to the illness that is typically characterized by fever, cough, and shortness of breath and may progress to pneumonia and respiratory failure. COVID-19 disease was first identified in Wuhan China in December 2019. In one embodiment, the patient with COVID-19 is confirmed by positive polymerase chain reaction (PCR) test (e.g. real time PCT. RT-PCT test) of a specimen (e.g., respiratory, blood, urine, stool, other bodily fluid specimen) from the patient. In one embodiment, the patient has SARS-CoV-2 specific antibodies (e.g. IgG and/or IgM antibodies), e.g. as determined by immunohistochemistry (IHC), enzyme-linked immunosorbent assay (ELISA), etc. Synonyms for COVID-19 include, without limitation, “novel coronavirus”, “2019 Novel Coronavirus” and “2019-nCoV”.

The term “patient” herein refers to a human patient. In one embodiment, the patient is hospitalized.

An “intravenous” or “iv” dose, administration, or formulation of a drug is one which is administered via a vein, e.g. by infusion.

A “subcutaneous” or “sc” dose, administration, or formulation of a drug is one which is administered under the skin, e.g. via a pre-filled syringe, auto-injector, or other device.

A “weight-based dose” of a drug refers to a dose that is based on the weight of the patient. In a preferred embodiment, where the drug is tocilizumab, the weight-based dose is 8 mg/kg (optionally ≤800 mg dose).

A “fixed dose” of a drug refers to a dose that is administered without regard to the patient's weight.

For the purposes herein, “clinical status” refers to a patient's health condition. Examples include that the patient is improving or getting worse. In one embodiment, clinical status is based on an ordinal scale of clinical status. In one embodiment, clinical status is not based on whether or not the patient has a fever.

An “ordinal scale of clinical status” refers to a scale used to quantify outcomes which are non-dimensional. They include can include an outcome at a single point in time or can examine change which has occurred between two points in time. In one embodiment, the two points of time are “Day 1” (when first dose, e.g. 8 mg/kg, of the IL-6 antagonist such as tocilizumab is administered) compared with “Day 28” (when the patient is evaluated) and, optionally, at “Day 60 (when the patient is further evaluated). Ordinal scales include various “categories” which each evaluate patent status or outcome. In one embodiment, the ordinal scale is a “7-category ordinal scale”.

In one embodiment, a “7-category ordinal scale” includes the following categories for evaluating the patient's status:
1. Discharged from hospital (or “ready for discharge”, e.g. as evidenced by normal body temperature and respiratory rate, and stable oxygen saturation on ambient air or ≤2 L supplemental oxygen)
2. Non-ICU hospital ward (or “ready for hospital ward”) not requiring supplemental oxygen
3. Non-ICU hospital ward (or -ready for hospital ward”) requiring supplemental oxygen
4. ICU or non-ICU hospital ward, requiring non-invasive ventilation or high-flow oxygen
5. ICU, requiring intubation and mechanical ventilation
6. ICU, requiring ECMO or mechanical ventilation and additional organ support (e.g. vasopressors, renal replacement therapy)
7. Death.

“Baseline” refers to a patient's status just prior to treatment and/or just prior to biomarker analysis. In one embodiment, the patient is not ventilated at baseline. In one embodiment, the patient is not receiving: a. continuous positive airway pressure (CPAP), b. bilevel positive airway pressure (BIPAP), or c. invasive ventilation at baseline. In one embodiment, the patient has SpO2<94% while on ambient air. In one embodiment, the patient does not have active bacterial, fungal, viral, or other infection (besides COVID-19) at baseline. In one embodiment, the patient has ALT or AST >5×ULN at baseline. In one embodiment, the patient has ANC <1000/mL at baseline. In one embodiment, the patient has platelet coune <50,000/mL at baseline.

For the purposes herein, “standard of care” or “SOC” refers to treatments or drugs commonly used to treat patients with pneumonia (e.g. viral pneumonia, such as COVID-19 pneumonia) including, inter alia, supportive care, administration of one or more anti-viral(s), and/or administration of one or more corticosteroid(s). In one embodiment, SOC comprises anti-viral (e.g. remdesivir or azithromycin) and/or corticosteroid (e.g. dexamethasone or prednisone) treatment.

“Supportive care” includes, without limitation: respiratory support (e.g. oxygen therapy via face mask or nasal cannula, high-flow nasal oxygen therapy or non-invasive mechanical ventilation, invasive mechanical ventilation, via extracorporeal membrane oxygenation (ECMO), etc.); circulation support (e.g. fluid resuscitation, boost microcirculation, vasoactive drugs); renal replacement therapy; plasma therapy; blood purification therapy; Xuebijing Injection (e.g. 100 mL/day twice a day); microecological preparation (e.g. probiotics, prebiotics, and synbiotics); anti-inflammatories (e.g. non-steroidal anti-inflammatory drugs, e.g. NSAIDs); herbal medicine; plasma (e.g. convalescent plasma) etc.

“Anti-viral” agents include, without limitation: alpha-interferon, lopinavir, ritonavir, lopinavir/ritonavir, remdesivir, azithromycin, ribavirin, hydroxychloroquine or chloroquine (with or without azithromycin), umifenovir, favipiravir etc. Optionally, the anti-viral is combined with alpha-interferon, ribavirin, and/or azithromycin. In one embodiment, the anti-viral is remdesivir or azithromycin.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone, such as methylprednisolone sodium succinate), dexamethasone or dexamethasone triamcinolone, hydrocortisone, and betamethasone. In one embodiment, the corticosteroid is selected from prednisone, methylprednisolone, hydrocortisone, and dexamethasone. In one embodiment, the corticosteroid is methylprednisolone. In one embodiment, the corticosteroid is “low-dose” glucocorticoid (e.g. ≤1-2 mg/kg/day methylprednisolone, e.g. for 3-5 days). In one embodiment, the corticosteroid is dexamethasone (e.g. oral or iv 6 mg once daily for up to 10 days) or prednisone.

An “anti-inflammatory” is a drug that reduces inflammation. Examples include, without limitation: steroids (e.g. dexamethasone), anti-ST2 (Astegolimab; MST1041A), IL-22Fc (UTTR1147A; see, e.g. US2014/0314711), statins, IL-6 antagonists, etc.

An “immunomodulator” is a drug that controls the immune system. Examples include, e.g., IL-6 antagonists, tocilizumab, sarilumab, anakinra, baricitinib, canakinumab, ruxolitinib, etc.

An “anti-coagulant” is a drug that helps prevent blood clots, e.g. heparin.

An “anti-fibrotic” is a drug that slows or halts fibrosis, e.g. tyrosine kinase inhibitor (e.g. imatinib) or pirfenidone.

An “anti-viral antibody” is one which binds to a virus and, preferably neutralizes the ability of the virus to infect a patient and/or replicate in a patient. In one embodiment, it comprises a cocktail of two or more anti-viral antibodies, e.g. REGN-COV2.

Herein “human interleukin 6” (abbreviated as “IL-6”) is a cytokine also known as B cell-stimulating factor 2 (BSF-2), or interferon beta-2 (IFNB2), hybridoma growth factor, and CTL differentiation factor. IL-6 was discovered as a differentiation factor contributing to activation of B cells (Hirano et al., Nature 324: 73-76 (1986)), and was later found to be a multifunction cytokine which influences the functioning of a variety of different cell types (Akira et al., Adv. in Immunology 54: 1-78 (1993)). Naturally occurring human IL-6 variants are known and included in this definition. Human IL-6 amino acid sequence information has been disclosed, see for example, www.uniprot.org/uniprot/P05231.

An “IL-6 antagonist” refers to agent that inhibits or blocks IL-6 biological activity via binding to human IL-6 or human IL-6 receptor. In one embodiment, the IL-6 antagonist is an antibody. In one embodiment, the IL-6 antagonist is an antibody that binds IL-6 receptor. Antibodies that bind IL-6 receptor include tocilizumab (including intravenous, iv, and subcutaneous sc formulations thereof) (Chugai, Roche, Genentech), satralizumab (Chugai, Roche, Genentech), sarilumab (Sanofi, Regeneron), NI-1201 (Novimmune and Tiziana), and vobarilizumab (Ablynx). In one embodiment, the IL-6 antagonist is a monoclonal antibody that binds IL-6. Antibodies that bind IL-6 include sirukumab (Centecor, Janssen), olokizumab (UCB), clazakizumab (BMS and Alder), siltuximab (Janssen), EBI-031 (Eleven Biotherapeutics and Roche). In one embodiment, the IL-6 antagonist is olamkicept.

For the purposes herein “human interleukin 6 receptor” (abbreviated as “IL-6R”) refers to the receptor which binds IL-6, including both membrane-bound IL-6R (mIL-6R) and soluble IL-6R (sIL-6R). IL-6R can combine with interleukin 6 signal transducer glycoprotein 130 to form an active receptor complex. Alternatively spliced transcript variants encoding distinct isoforms of IL-6 have been reported and are included in this definition. The amino acid sequence structure of human IL-6R and its extracellular domain have been described; see, for example, Yamasaki et al., Science, 241: 825 (1988).

A “neutralizing” anti-IL-6R antibody herein is one which binds to IL-6R and is able to inhibit, to a measurable extent, the ability of IL-6 to bind to and/or active IL-6R. Tocilizumab is an example of a neutralizing anti-IL-6R antibody.

“Tocilizumab” or “TCZ” is a recombinant humanized monoclonal antibody that binds to human interleukin-6 receptor (IL-6R). It is an IgG1κ (gamma 1, kappa) antibody with a two heavy chains and two light chains forming two antigen-binding sites. In a preferred embodiment, the light chain and heavy chain amino acid sequences of Tocilizumab comprise SEQ ID NOs. 1 and 2, respectively.

A “native sequence” protein herein refers to a protein comprising the amino acid sequence of a protein found in nature, including naturally occurring variants of the protein. The term as used herein includes the protein as isolated from a natural source thereof or as recombinantly produced.

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

“Antibody fragments” herein comprise a portion of an intact antibody which retains the ability to bind antigen. Examples of antibody fragments include Fab, Fab′, F(ab′)₂, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature. 352:624-628 (1991) and Marks et al., J. Mot. Biol., 222:581-597 (1991), for example. Specific examples of monoclonal antibodies herein include chimeric antibodies, humanized antibodies, and human antibodies, including antigen-binding fragments thereof.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). Humanized antibodies herein specifically include “reshaped” IL-6R antibodies as described in U.S. Pat. No. 5,795,965, expressly incorporated herein by reference.

A “human antibody” herein is one comprising an amino acid sequence structure that corresponds with the amino acid sequence structure of an antibody obtainable from a human B-cell, and includes antigen-binding fragments of human antibodies. Such antibodies can be identified or made by a variety of techniques, including, but not limited to: production by transgenic animals (e.g., mice) that are capable, upon immunization, of producing human antibodies in the absence of endogenous immunoglobulin production (see, e.g., Jakobovits et al., Proc. Natl. Acad. Sci. USA, 90:2551 (1993); Jakobovits et al., Nature. 362:255-258 (1993); Bruggermann et al., Year in Immuno., 7:33 (1993); and U.S. Pat. Nos. 5,591,669, 5,589,369 and 5,545,807)): selection from phage display libraries expressing human antibodies or human antibody fragments (see, for example, McCafferty et al., Nature 348:552-553 (1990); Johnson et al., Current Opinion in Structural Biology 3:564-571 (1993); Clackson et al., Nature, 352:624-628 (1991); Marks et al., J. Mol. Biol. 222:581-597 (1991); Griffith et al., EMBO J. 12:725-734 (1993); U.S. Pat. Nos. 5,565,332 and 5,573,905); generation via in vitro activated B cells (see U.S. Pat. Nos. 5,567,610 and 5,229,275); and isolation from human antibody producing hybridomas.

A “multispecific antibody” herein is an antibody having binding specificities for at least two different epitopes. Exemplary multispecific antibodies may bind to two different epitopes of IL-6R. Alternatively, an anti-IL-6R binding arm may be combined with an arm that binds to a triggering molecule on a leukocyte such as a T-cell receptor molecule (e. g. CD2 or CD3), or Fc receptors for IgG (FcγR), such as FcγRI (CD64), FcγRII (CD32) and FcγRIII (CD16) so as to focus cellular defense mechanisms to the receptor. Multispecific antibodies can be prepared as full-length antibodies or antibody fragments (e.g. F(ab′)₂ bispecific antibodies). Engineered antibodies with three or more (preferably four) functional antigen binding sites are also contemplated (see, e.g., US Appln. No. US 2002/0004587 A1, Miller et al.).

Antibodies herein include “amino acid sequence variants” with altered antigen-binding or biological activity. Examples of such amino acid alterations include antibodies with enhanced affinity for antigen (e.g. affinity matured antibodies), and antibodies with altered Fc region, if present, e.g. with altered (increased or diminished) antibody dependent cellular cytotoxicity (ADCC) and/or complement dependent cytotoxicity (CDC) (see, for example, WO 00/42072, Presta, L. and WO 99/51642, Iduosogie et al.); and/or increased or diminished serum half-life (see, for example, WO00/42072, Presta, L.).

The antibody herein may be conjugated with a “heterologous molecule” for example to increase half-life or stability or otherwise improve the antibody. For example, the antibody may be linked to one of a variety of non-proteinaceous polymers, e.g., polyethylene glycol (PEG), polypropylene glycol, polyoxyalkylenes, or copolymers of polyethylene glycol and polypropylene glycol. Antibody fragments, such as Fab′, linked to one or more PEG molecules are an exemplary embodiment of the invention.

The antibody herein may be a “glycosylation variant” such that any carbohydrate attached to the Fc region, if present, is altered. For example, antibodies with a mature carbohydrate structure that lacks fucose attached to an Fc region of the antibody are described in US Pat Appl No US 2003/0157108 (Presta, L.). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in WO 2003/011878, Jean-Mairet et al. and U.S. Pat. No. 6,602,684. Umana et al. Antibodies with at least one galactose residue in the oligosaccharide attached to an Fc region of the antibody are reported in WO 1997/30087, Patel et al. See, also, WO 1998/58964 (Raju, S.) and WO 1999/22764 (Raju, S.) concerning antibodies with altered carbohydrate attached to the Fe region thereof. See also US 2005/0123546 (Umana et al.) describing antibodies with modified glycosylation.

The term “hypervariable region” when used herein refers to the amino acid residues of an antibody that are responsible for antigen binding. The hypervariable region comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined. The hypervariable regions of Tocilizumab comprise:

L1-
(SEQ ID NO: 3)
Arg Ala Ser Gln Asp Ile Ser Tyr Leu Asn;
L2-
(SEQ ID NO: 4)
Tyr Thr Ser Arg Leu His Ser;
L3-
(SEQ ID NO: 5)
Gln Gly Asn Thr Leu Pro Tyr Thr;
H1-
(SEQ ID NO: 6)
Ser Asp His Ala Trp Ser;
H2-
(SEQ ID NO: 7)
Tyr Ile Ser Tyr Ser Gly Ile Thr Tyr Asn
Pro Ser Leu Lys Ser;
and
H3-
(SEQ ID NO: 8)
Ser Leu Ala Arg Thr Ala Met Asp Tyr.

In one embodiment herein, the IL-6R antibody comprises the hypervariable regions of Tocilizumab.

A “full length antibody” is one which comprises an antigen-binding variable region as well as a light chain constant domain (CL) and heavy chain constant domains, CH1, CH2 and CH3. The constant domains may be native sequence constant domains (e.g. human native sequence constant domains) or amino acid sequence variants thereof. Preferably, the full length antibody has one or more effector functions. Tocilizumab is an example of a full-length antibody.

A “naked antibody” is an antibody (as herein defined) that is not conjugated to a heterologous molecule, such as a cytotoxic moiety, polymer, or radiolabel.

Antibody “effector functions” refer to those biological activities attributable to the Fc region (a native sequence Fc region or amino acid sequence variant Fc region) of an antibody. Examples of antibody effector functions include C1q binding, complement dependent cvtotoxicity (CDC), Fc receptor binding, antibody-dependent cell-mediated cytotoxicity (ADCC), etc.

Depending on the amino acid sequence of the constant domain of their heavy chains, full length antibodies can be assigned to different “classes”. There are five major classes of full length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known.

The term “recombinant antibody”, as used herein, refers to an antibody (e.g. a chimeric, humanized, or human antibody or antigen-binding fragment thereof) that is expressed by a recombinant host cell comprising nucleic acid encoding the antibody. Examples of “host cells” for producing recombinant antibodies include: (1) mammalian cells, for example, Chinese Hamster Ovary (CHO), COS, myeloma cells (including Y0 and NS0 cells), baby hamster kidney (BHK), Hela and Vero cells: (2) insect cells, for example, sf9, sf21 and Tn5; (3) plant cells, for example plants belonging to the genus Nicotiana (e.g. Nicotiana tabacum); (4) yeast cells, for example, those belonging to the genus Saccharomyces (e.g. Saccharomyces cerevisiae) or the genus Aspergillus (e.g. Aspergillus niger); (5) bacterial cells, for example Escherichia coli cells or Bacillus subtilus cells, etc.

As used herein. “specifically binding” or “binds specifically to” refers to an antibody selectively or preferentially binding to IL-6R antigen. Preferably the binding affinity for antigen is of Kd value of 10⁻⁹ mol/l or lower (e.g. 10⁻¹⁰ mol/l), preferably with a Kd value of 10⁻¹⁰ mol/l or lower (e.g. 10⁻¹² mol/l). The binding affinity is determined with a standard binding assay, such as surface plasmon resonance technique (BIACORE®).

Examples of “non-steroidal anti-inflammatory drugs” or “NSAIDs” include aspirin, acetylsalicylic acid, ibuprofen, flurbiprofen, naproxen, indomethacin, sulindac, tolmetin, phenylbutazone, diclofenac, ketoprofen, benorylate, mefenamic acid, methotrexate, fenbufen, azapropazone; COX-2 inhibitors such as celecoxib (CELEBREX®; 4-(5-(4-methylphenyl)-3-(trifluoromethyl)-1H-pyrazol-1-yl) benzenesulfonamide, valdecoxib (BEXTRA®), meloxicam (MOBIC®), GR 253035 (Glaxo Wellcome); and MK966 (Merck Sharp & Dohme), including salts and derivatives thereof, etc. Specific embodiments include: aspirin, naproxen, ibuprofen, indomethacin, and tolmetin.

Regarding an IL-6 antagonist, an “effective amount” refers to an amount of the IL-6 antagonist (e.g. IL-6 receptor antibody such as tocilizumab) that is effective for treating pneumonia (e.g. viral pneumonia, including COVID-19 pneumonia) and/or for treating acute respiratory distress syndrome (ARDS).

The term “pharmaceutical formulation” refers to a preparation which is in such form as to permit the biological activity of the active ingredient or ingredients to be effective, and which contains no additional components which are unacceptably toxic to a subject to which the formulation would be administered. Such formulations are sterile. In one embodiment, the formulation is for intravenous (iv) administration. In another embodiment, the formulation is for subcutaneous (sc) administration.

A “sterile” formulation is aseptic or free from all living microorganisms and their spores.

A “liquid formulation” or “aqueous formulation” according to the invention denotes a formulation which is liquid at a temperature of at least about 2 to about 8° C.

The term “lyophilized formulation” denotes a formulation which is dried by freezing the formulation and subsequently subliming the ice from the frozen content by any freeze-drying methods known in the art, for example commercially available freeze-drying devices. Such formulations can be reconstituted in a suitable diluent, such as water, sterile water for injection, saline solution etc., to form a reconstituted liquid formulation suitable for administration to a subject.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

An “elevated” level of a biomarker refers to an amount of that biomarker in the patient that is above the upper limit of normal (ULN).

An “elevated IL-6 level” is ≥15 pg/mL, or ≥10 pg/mL or >7 pg/mL, e.g. as measured by enzyme linked immunosorbent assay (ELISA) of a blood sample from the patient. In one embodiment, “normal” IL-6 level is considered to be 7 pg/mL. In one embodiment, elevated IL-6 level is ? 80 ng/L, e.g. as measured by ELISA.

The patient who has “not been found to have elevated IL-6 levels by laboratory testing” has been treated according to the methods herein without regard to his or her IL-6 level. In one embodiment, such patient does not have an elevated IL-6 level.

“Remdesivir” is an antiviral medication, a nucleotide analog, specifically an adenosine analogue, which inserts into viral RNA chains, causing their premature termination. Its molecular formula is C₂₇H₃₅N₆O₈P and IUPAC Name is 2-ethylbutyl (2S)-2-[[[(2R,3S,4R,5R)-5-(4-aminopyrrolo[2,1-f][1,2,4]triazin-7-yl)-5-cyano-3,4-dihydroxyoxolan-2-yl]methoxy-phenoxyphosphoryl]amino]propanoate. Remdesivir's laboratory name is GS-5734 and its CAS number is 1809249-37-3. It is described in U.S. Pat. No. 9,724,360 and is manufactured by Gilead Sciences.

The term “biomarker” as used herein refers to an indicator, e.g., predictive, diagnostic, and/or prognostic, which can be detected in a sample, for example, ferritin and IL-6 biomarkers. Preferably the biomarker is predictive of patient response to an IL-6 antagonist. Biomarkers include, but are not limited to, polynucleotides (e.g., DNA and/or RNA), polynucleotide copy number alterations (e.g., DNA copy numbers), polypeptides, polypeptide and polynucleotide modifications (e.g., post-translational modifications), carbohydrates, and/or glycolipid-based molecular markers. In one embodiment the biomarker is ferritin. In one embodiment, the biomarker is IL-6.

The “amount” or “level” of a biomarker associated with an increased clinical benefit to an individual is a detectable level in a biological sample. These can be measured by methods known to one skilled in the art and also disclosed herein. The expression level or amount of biomarker assessed can be used to determine the response to the treatment.

A “level above the upper limit of normal” refers to an amount of a biomarker that is abnormal or atypical in a subject (including a healthy subject) or patient (including one with pneumonia or experiencing inflammation). Assays for measuring such abnormal amounts of ferritin and IL-6 are known in the art and disclosed herein, along with exemplary “cut-offs” or “comparator” amounts of ferritin or IL-6 for identifying patients eligible for therapy.

The term “sample,” as used herein, refers to a composition that is obtained or derived from a subject or patient of interest that contains a cellular and/or other molecular entity that is to be characterized and/or identified. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In one embodiment, the sample is a blood specimen from the patient. In one embodiment, the sample is a serum sample from the patient. In one embodiment, the sample is a plasma sample from the patient.

“Ferritin” is a protein that stores and releases iron in the body. For the purposes herein, “ferritin” refers to human ferritin. Ferritin is a globular protein complex comprising 24 protein subunits forming a nanocage with multiple metal-protein interactions.

“Ferritin level” can be measured in a sample from a patient or subject, e.g. a blood sample (whole blood, serum, and/or plasma) using assays which are standard in the field. Exemplary ferritin assays include, without limitation: labelled nonradiometric assays (e.g. EIA, enzyme immunoassay; fluorimetric assay; ELISA: Enzyme linked immunosorbent assay: chemiluminescent assay (e.g. ECL: ElectroChemiLuminescence assay, e.g. Roche ELECSYS® assay); MEIA: microparticle enzyme immunoassay; RPIA: Radial partition immunoassay); labelled radiometric assays (e.g. RIA: Radioimmune assay; IRMA: Immunoradiometric assay); agglutination assays (e.g. Turbidimetric assay; Nephelometric assay: LPIA: Latex photometric immunoassay); see, for example, Garcia-Casel et al. PLoS One. 2018; 13(5): e0196576. In one embodiment, the assay is an enzyme immunoassay or chemiluminescent assay. In one embodiment, the patient sample is a serum or plasma sample.

For the purposes herein “normal ferritin level” refers to the ferritin level in a normal (male or female) subject who is not ferritin deficient or who is not experiencing inflammation resulting in elevated ferritin level. In general, normal ferritin levels range from about 12 to about 300 nanograms per milliliter of blood (ng/mL) for males and about 12 to about 150 ng/mL for females. See, for example, www.medicinenet.com/ferritin_blood_test/article.htm.

By “elevated”, “abnormally high”, or “higher-than-normal” ferritin level herein is meant an amount of ferritin that is higher than the “upper normal” ferritin level in a subject, for example >300 ng/mL or 400 ng/mL for male patient, >150 ng/mL for female patient, ≥about 2198 pmol/L or ≥about 3150 pmol/L, e.g., measured using enzyme immunoassay or chemiluminescent assay (e.g. Elecsys® Ferritin assay).

### II. Production of IL-6 Antagonists

IL-6 antagonists contemplated herein include antagonists that bind to IL-6 or IL-6 receptor.

In one embodiment, the IL-6 antagonist is an antibody.

In one embodiment, the IL-6 antagonist is an antibody that binds IL-6 receptor.

In one embodiment, the IL-6 antagonist is an antibody that binds to both membrane-bound IL-6 receptor and soluble IL-6 receptor.

In one embodiment, the IL-6 antagonist blocks the IL-6/IL-6 receptor complex as well as depleting circulating levels of IL-6 in the blood.

Antibodies that bind IL-6 receptor include tocilizumab (including intravenous, iv, and subcutaneous sc formulations thereof) (Chugai, Roche, Genentech), satralizumab (Chugai, Roche, Genentech), sarilumab (Sanofi, Regeneron), NI-1201 or TZLS-501 (Novimmune and Tiziana), and vobarilizumab (Ablynx).

In one embodiment, the IL-6 antagonist is tocilizumab.

Tocilizumab, also named Myeloma Receptor Antibody (MRA), is a recombinant humanized monoclonal antibody that selectively binds to human interleukin-6 receptor (IL-6R). It is an IgG1κ (gamma 1, kappa) antibody with a typical H₂L₂ structure. The tocilizumab molecule is composed of two heterodimers. Each of the heterodimers is composed of a heavy (H) and a light (L) polypeptide chain. The four polypeptide chains are linked intra- and inter-molecularly by disulfide linkages. The molecular formula and theoretical molecular weight of the tocilizumab antibody are as follows:

Molecular formula: C₆₄₂₈H₉₉₇₆N₁₇₂₀O₂₀₁₈S₄₂ (polypeptide moiety only)

Molecular weight: 144.985 Da (polypeptide moiety only).

The amino acid sequence of the light chain deduced from complimentary deoxyribonucleic acid (cDNA) sequences and confirmed by liquid chromatography mass-spectrometry (LC-MS) peptide mapping is in SEQ ID Nos. 1 and 2. The five light chain cysteine residues of each heterodimer are involved in two intrachain disulfide linkages and one interchain disulfide linkage:

Intrachain linkages: Cys_{L23}-Cys_{L88} and Cys_{L134}-Cys_{L194}

Linkage between heavy and light chain: Cys_{L214} and Cys_{H222}

Assignments of the disulfide linkages are based on sequence homology to other IgG1 antibodies and were confirmed by liquid chromatography mass-spectrometry (LC-MS) peptide mapping performed using material from the fourth generation (G4) process. Cys_{Lx} and Cys_{Hx} denote cysteine residues at position x of the light and heavy chains, respectively.

| Amino Acid Sequence of the L Chain of the Tocilizumab Molecule | | |
|---|---|---|
| SEQ ID NO. 1 | | |
| 1 | DIQMTQSPSS LSASVGDRVT ITCRASQDIS SYLNWYQQKP GKAPKLLIYY | 50 |
| 51 | TSRLHSGVPS RESGSGSGTD FTFTISSLQP EDIATYYCQQ GNTLPYTFGQ | 100 |
| 101 | GTKVEIKRTV AAPSVFIFPP SDEQLKSGTA SVVCLLNNFY PREAKVQWKV | 150 |
| 151 | DNALQSGNSQ ESVTEQDSKD STYSLSSTLT LSKADYEKHK VYACEVTHQG | 200 |
| 201 | LSSPVTKSFN RGEC | 214 |

Note:
The entire sequence has been determined by LC-MS peptide mapping.

The amino acid sequence of the heavy chain deduced from complimentary deoxyribonucleic acid (cDNA) sequences and confirmed by amino acid sequencing is in SEQ ID NO. 2. The eleven heavy chain cysteine residues of each heterodimer are involved in four intrachain disulfide linkages, two interchain disulfide linkages between the two heavy chains and the third interchain disulfide linkage between the heavy chain and the light chain of each of the heterodimers:

Intrachain linkages: Cys_{H22}-Cys_{H96}, CLS_{H146}-Cys_{H202}, CyS_{H263}-Cys_{H323} and Cys_{H369}-Cys_{H142}

Linkages between the two heavy chains: Cys_{H228}-Cys_{H228} and Cys_{H231}-Cys_{H231}

Linkage between heavy and light chain: Cys_{L214}-Cys_{H222}

Assignments of the disulfide linkages are based on sequence homology to other IgG1 antibodies and were confirmed by LC-MS peptide mapping performed using material from the G4 process.

| Amino Acid Sequence of the H Chain of the Tocilizumab Molecule | |
|---|---|
| SEQ ID NO. 2 | |
| 1 | pEVQLQESGPG LVRPSQTLSL TQTVSGYSIT SDHAWSWVRQ PPGRGLEWIG 50 |
| 51 | YISYSGITTY NPSLKSRVTM LRDTSKNQFS LELSSVTAAD TAVYYCARSL 100 |
| 101 | ARTTAMDYWG QGSLVTVSSA STKGPSVFPL APSSKSTSGG TAALGCLVKD 150 |
| 151 | YFPEPVIVSW NSGALTSGVH TFPAVLQSSG LYSLSSVVTV PSSSLGTQTY 200 |
| 201 | ICNVNHKPSN TKVDKKVEPK SCDKTHTCPP CPAPELLGGP SVFLFPPKPK 250 |
| 251 | DTLMISRTPE VICVVVDVSH EDPEVKFNWY VDGVEVHNAK TKPREEQYNS 300 |
| 301 | TYRVVSVLTV LHQDWLNGKE YKCKVSNKAL PAPIEKTISK AKGQPREPQV 350 |
| 351 | YTLPPSRDEL TKNQVSLTCL VKGFYPSDIA VEWESNGQPE NNYKTTPPVL 400 |
| 401 | DSDGSFFLYS KLTVDKSRWQ QGNVFSCSVM HEALHNHYTQ KSLSLSPG 448 |

Note:
The entire sequence has been determined by LC-MS peptide mapping.
The N-terminus of the heavy chain has been determined to be
predominantly a pyroglutamic acid residue (pE).

In one embodiment, the IL-6 antagonist is satralizumab. Satralizumab (also called SA237) is a humanized monoclonal antibody that binds IL-6 receptor See U.S. Pat. No. 8,562,991.

In one embodiment, the IL-6 antagonist is the human antibody that binds the IL-6 receptor called TZLS-501 (Tiziana) or N1-1201 (Novimmune).

In one embodiment, the IL-6 antagonist is a monoclonal antibody that binds IL-6.

Antibodies that bind IL-6 include sirukumab (Centecor, Janssen), olokizumab (UCB), clazakizumab (BMS and Alder), siltuximab (Janssen), EBI-031 (Eleven Biotherapeutics and Roche).

In one embodiment, the IL-6 antagonist is olamkicept. Olamkicept is a recombinant protein that fuses the extracellular domain of the signal transducing subunit of the IL-6 receptor, IL-6Rβ (glycoprotein 130, gp130), to a human IgG Fc fragment. The full construct is a dimer of covalently linked identical peptide chains. Mechanistically olamkicept acts as an inhibitor of the IL-6 signaling pathway. Olamkicept inhibits trans-signaling by the soluble IL-6 receptor (sIL-6R).

In a preferred embodiment, the methods and articles of manufacture of the present invention use, or incorporate, an antibody that binds to human IL-6R. IL-6R antigen to be used for production of, or screening for, antibodies may be, e.g., a soluble form of IL-6R or a portion thereof (e.g. the extracellular domain), containing the desired epitope. Alternatively, or additionally, cells expressing IL-6R at their cell surface can be used to generate, or screen for, antibodies. Other forms of IL-6R useful for generating antibodies will be apparent to those skilled in the art.

In one embodiment, the antibody is an antibody fragment, various such fragments being disclosed above.

In another embodiment, the antibody is an intact or full-length antibody. Depending on the amino acid sequence of the constant domain of their heavy chains, intact antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called α, δ, ε, γ, and μ, respectively. The subunit structures and three-dimensional configurations of different classes of immunoglobulins are well known. In a preferred embodiment, the anti-IL-6R antibody is an IgG1 or IgM antibody.

Techniques for generating antibodies are known and examples provided above in the definitions section of this document. In a preferred embodiment, the antibody is a chimeric, humanized, or human antibody or antigen-binding fragment thereof. Preferably the antibody is a humanized full-length antibody.

Various techniques are available for determining binding of the antibody to the IL-6R. One such assay is an enzyme linked immunosorbent assay (ELISA) for confirming an ability to bind to human IL-6R. See, for example, U.S. Pat. No. 5,795,965. According to this assay, plates coated with IL-6R (e.g. recombinant sIL-6R) are incubated with a sample comprising the anti-IL-6R antibody and binding of the antibody to the sIL-6R is determined.

Preferably, the anti-IL-6R antibody is neutralizes IL-6 activity, e.g. by inhibiting binding of IL-6 to IL-6R. An exemplary method for evaluating such inhibition is disclosed in U.S. Pat. Nos. 5,670,373, and 5,795,965, for example. According to this method, the ability of the antibody to compete with IL-6 to IL-6R is evaluated. For example, a plate is coated with IL-6R (e.g. recombinant sIL-6R), a sample comprising the anti-IL-6R antibody with labeled IL-6 is added, and the ability of the antibody to block binding of the labeled IL-6 to the IL-6R is measured. See, U.S. Pat. No. 5,795,965. Alternatively, or additionally, identification of binding of IL-6 to membrane-bound IL-6R is carried out according to the method of Taga et al. J. Exp. Med., 166: 967 (1987). An assay for confirming neutralizing activity using the IL-6-dependent human T-cell leukemia line KT3 is also available, see, U.S. Pat. No. 5,670,373, and Shimizu et al. Blood 72: 1826 (1988).

Non-limiting examples of anti-IL-6R antibodies herein include PM-1 antibody (Hirata et al., J Immunol. 143:2900-2906 (1989). AUK12-20, AUK64-7, and AUK146-15 antibody (U.S. Pat. No. 5,795,965), as well as humanized variants thereof, including, for example tocilizumab. See, U.S. Pat. No. 5,795,965. Preferred examples of the reshaped human antibodies used in the present invention include humanized or reshaped anti-interleukin (IL-6) receptor antibodies (hPM-1 or MRA) (see U.S. Pat. No. 5,795,965).

The antibody herein is preferably recombinantly produced in a host cell transformed with nucleic acid sequences encoding its heavy and light chains (e.g. where the host cell has been transformed by one or more vectors with the nucleic acid therein). The preferred host cell is a mammalian cell, most preferably a Chinese Hamster Ovary (CHO) cells.

### III. Pharmaceutical Formulations

Therapeutic formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine: preservatives (such as octadecyldimethylbenzyl ammonium chloride: hexamethonium chloride; benzalkonium chloride, benzethonium chloride; phenol, butyl or benzyl alcohol: alkyl parabens such as methyl or propyl paraben; catechol; resorcinol; cyclohexanol; 3-pentanol; and m-cresol); low molecular weight (less than about 10 residues) polypeptides: proteins, such as serum albumin, gelatin, or immunoglobulins: hydrophilic polymers such as polyvinylpyrrolidone; amino acids such as glycine, glutamine, asparagine, histidine, arginine, or lysine: monosaccharides, disaccharides, and other carbohydrates including glucose, mannose, or dextrins: chelating agents such as EDTA; sugars such as sucrose, mannitol, trehalose or sorbitol; salt-forming counter-ions such as sodium: metal complexes (e.g. Zn-protein complexes); and/or non-ionic surfactants such as TWEEN™, PLURONICS™ or polyethylene glycol (PEG).

The formulation herein may also contain more than one active compound as necessary, preferably those with complementary activities that do not adversely affect each other. The type and effective amounts of such medicaments depend, for example, on the amount of antibody present in the formulation, and clinical parameters of the subjects. Exemplary such medicaments are discussed below.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition. Osol, A. Ed. (1980).

Sustained-release preparations may be prepared. Suitable examples of sustained-release preparations include semi-permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

In one embodiment, the formulation is suitable for intravenous (iv) infusion, for example, the tocilizumab iv formulation as disclosed in U.S. Pat. Nos. 8,840,884 and 9,051,384. In one embodiment, a tocilizumab iv formulation is a sterile, clear, colorless to pale yellow, preservative-free solution for further dilution prior to intravenous infusion with a pH of approximately 6.5. In one embodiment, a tocilizumab iv formulation is supplied in a single-dose vial, formulated with a disodium phosphate dodecahydrate/sodium dihydrogen phosphate dihydrate buffered solution, and is available at a concentration of 20 mg/mL containing 80 mg/4 mL, 200 mg/10 mL, or 400 mg/20 mL of tocilizumab. In one embodiment. each mL of tocilizumab iv solution contains polysorbate 80 (0.5 mg), sucrose (50 mg), and Water for Injection, USP.

In one embodiment, the formulation is suitable for subcutaneous (sc) administration, for example, the tocilizumab sc formulation as in U.S. Pat. No. 8,568,720. In one embodiment, a tocilizumab sc formulation is a sterile, clear, colorless to slightly yellowish, preservative-free, histidine buffered solution for subcutaneous use with a pH of approximately 6.0. In one embodiment, a tocilizumab sc formulation is supplied in a ready-to-use, single-dose 0.9 mL prefilled syringe (PFS) with a needle safety device, or a ready-to-use, single-dose 0.9 mL autoinjector. In one embodiment tocilizumab sc formulation delivers 162 mg tocilizumab, L-arginine hydrochloride (19 mg), L-histidine (1.52 mg), L-histidine hydrochloride monohydrate (1.74 mg). L-methionine (4.03 mg), polysorbate 80 (0.18 mg), and Water for Injection.

### IV. Diagnostic Methods

In one embodiment, the invention provides a method of identifying a patient having pneumonia who may benefit from a treatment with an IL-6 antagonist, the method comprising measuring ferritin level in a sample from the patient, wherein an elevated ferritin level identifies the patient as one who will benefit from the treatment.

Ferritin level can be measured in a sample from a patient or subject. Preferably, the sample is a blood sample, e.g. whole blood, serum, or plasma, with serum or plasma samples being preferred.

Exemplary ferritin assays include, without limitation: labelled nonradiometric assays (e.g. EIA, enzyme immunoassay; fluorimetric assay; ELISA: Enzyme linked immunosorbent assay; chemiluminescent assay (e.g. ECL: ElectroChemiLuminescence assay, e.g. Roche ELECSYS, assay): MEIA: microparticle enzyme immunoassay; RPIA: Radial partition immunoassay); labelled radiometric assays (e.g. RIA: Radioimmune assay; IRMA: Immunoradiometric assay); agglutination assays (e.g. Turbidimetric assay: Nephelometric assay: LPIA: Latex photometric immunoassay); see, for example, Garcia-Casel et al. PLoS One. 2018; 13(5): e0196576.

In one embodiment the ferritin assay is an enzyme immunoassay.

In one embodiment, the ferritin assay is a chemiluminescent assay.

In one embodiment, the ferritin assay is an ElectroChemiLuminescence (ECL) assay, e.g. the Roche ELECSYS® assay.

In one embodiment, the ferritin level in the sample is elevated, abnormally high, higher-than-normal, or higher than the upper normal ferritin level in a subject.

In one embodiment, the ferritin level is >300 ng/mL or >400 ng/mL for a male patient.

In one embodiment, the ferritin level is >150 mg/mL for a female patient.

In one embodiment, the ferritin level is ≥about 2198 pmol/L.

In one embodiment, the ferritin level is ≥about 3150 pmol/L.

In another embodiment, the invention provides a method of identifying a hospitalized patient having pneumonia who is receiving non-invasive ventilation or high flow oxygen or who is intubated and being mechanically ventilated who may benefit from treatment with an IL-6 antagonist, the method comprising measuring IL-6 level in a sample from the patient, wherein an elevated IL-6 level identifies the patient as one who will benefit from shortened time to hospital discharge.

IL-6 level can be measured in a sample from a patient or subject. Preferably, the sample is a blood sample, e.g. whole blood, serum, plasma, or combinations thereof, with serum or plasma samples being preferred.

In one embodiment, the expression level of IL-6 in a sample from the individual has been determined to be above a reference IL-6 expression level, e.g. wherein the reference IL-6 expression level is a pre-assigned IL-6 expression level. For example, the expression level of IL-6 in the sample is an expression level of IL-6 that is at least four standard deviations above the reference IL-6 expression level.

In one embodiment, the expression level of IL-6 in the sample is a protein expression level of IL-6, e.g. by enzyme linked immunosorbent assay (ELISA).

In one embodiment, the expression level of IL-6 is an mRNA expression level of IL-6. Assays for measuring mRNA expression level of IL-6 include in situ hybridization (ISH) (e.g. using a probe targeting nucleotides 2-1082 of an IL-6 mRNA), RNA-seq, RT-qPCR, qPCR, multiplex qPCR or RT-qPCR, microarray analysis, SAGE, MassARRAY technique, FISH, or a combination thereof.

In one embodiment, the elevated IL-6 level is ≥15 pg/mL, e.g. as measured by ELISA.

In one embodiment, the elevated IL-6 level is ≥10 pg/mL, e.g. as measured by ELISA.

In one embodiment, elevated IL-6 level is ≥80 ng/L, e.g. as measured by ELISA.

### V. Therapeutic Uses of Anti-IL-6 Antagonists

In one embodiment, the invention concerns a method of treating pneumonia in a patient who is not mechanically ventilated comprising administering an IL-6 antagonist to the patient in an amount effective to prevent the patient from dying or receiving mechanical ventilation.

In one embodiment, the effective amount prevents the patient from receiving mechanical ventilation.

In one embodiment, the pneumonia is viral pneumonia.

In one embodiment, the pneumonia is moderate, severe, or critical pneumonia.

In one embodiment, the pneumonia is severe pneumonia.

In one embodiment, the pneumonia is coronavirus pneumonia.

In one embodiment, the pneumonia is COVID-19 pneumonia, Middle East respiratory syndrome (MERS-CoV) pneumonia, or severe acute respiratory syndrome (SARS-CoV) pneumonia.

In one embodiment, the pneumonia is COVID-19 pneumonia.

In one embodiment, the patient is hospitalized.

In one embodiment, the patient has hypoxia.

In one embodiment, the effective amount of the IL-6 antagonist prevents the patient from dying or receiving mechanical ventilation by about Day 28 from a first administration of the IL-6 antagonist or from baseline.

In one embodiment, administration of the IL-6 antagonist prevents the patient from dying or receiving mechanical ventilation within about 28 days from a first administration of the IL-6 antagonist.

In one embodiment, wherein the estimated cumulative proportion of patients with death or mechanical ventilation rate at Day 28 is about 12% with IL-6 antagonist administration compared with about 19% in patients not receiving the anti-IL-6 antagonist.

In one embodiment, the patient further receives standard of care.

In one embodiment, treatment with the IL-6 antagonist reduces the risk of the patient dying or receiving mechanical ventilation compared to the patient not receiving the IL-6 antagonist but receiving standard of care (SOC).

In one embodiment, administration of the IL-6 antagonist reduces the risk of death or requiring mechanical ventilation by at least about 40% (e.g. about 44%), e.g. compared to the patient to whom is administered the SOC without the IL-6 antagonist.

In one embodiment, the SOC comprises administration of anti-viral (e.g. remdesiver) and/or administration of corticosteroid (e.g. dexamethasone).

In one embodiment, the effective amount of the IL-6 antagonist further reduces likelihood of clinical failure, e.g. wherein clinical failure comprises time to: death. mechanical ventilation, ICU admission, and/or 2-category worsening in the 7-category ordinal scale wherein the patient was already admitted to the ICU prior to treatment. According to this embodiment, the method optionally reduces the risk of clinical failure up to Day 28.

In another aspect, the invention concerns a method of treating viral pneumonia in a patient comprising who is not mechanically ventilated comprising administering a combination of an IL-6 antagonist and remdesivir to the patient in an amount effective to prevent the patient from dying or receiving mechanical ventilation.

According to this embodiment, the IL-6 antagonist is preferably tocilizumab.

According to this embodiment, the effective amount of tocilizumab comprises a first weight-based 8 mg/kg intravenous dose of tocilizumab optionally followed by a second weight-based 8 mg/kg intravenous dose of tocilizumab 8-24 hours after the first dose.

According to this embodiment, the effective amount of remdesivir comprises an initial one-time dose of 200 mg followed by 100 mg per day, and wherein 5 to 10 total doses of remdesivir are administered to the patient.

In another embodiment, the invention concerns a method of treating viral pneumonia in a patient who is not mechanically ventilated comprising administering a combination of an IL-6 antagonist and a corticosteroid to the patient in an amount effective to prevent the patient from dying or receiving mechanical ventilation.

According to this embodiment, the effective amount of tocilizumab preferably comprises a first weight-based 8 mg/kg intravenous dose of tocilizumab optionally followed by a second weight-based 8 mg/kg intravenous dose of tocilizumab 8-24 hours after the first dose.

According to this embodiment, wherein the corticosteroid preferably comprises dexamethasone (e.g. administered orally or intravenously 6 mg once daily for up to 10 days).

In one embodiment, the patient treated herein is identified as having elevated ferritin level.

In one embodiment, the patient treated herein has elevated IL-6 level.

According to certain embodiments, the pneumonia is:
viral pneumonia;
moderate pneumonia;
moderate-severe pneumonia;
severe pneumonia;
severe-critical pneumonia;
critical pneumonia;
coronavirus pneumonia;
COVID-19 pneumonia;
Middle East respiratory syndrome (MERS-CoV) pneumonia;
severe acute respiratory syndrome (SARS-CoV) pneumonia
severe COVID-19 pneumonia;
critical COVID-19 pneumonia;
moderate COVID-19 pneumonia;
moderate-severe COVID-19 pneumonia;
severe-critical COVID-19 pneumonia;
viral pneumonia with hypoxia;
hospitalized pneumonia;
hospitalized pneumonia with hypoxia;
hospitalized COVID pneumonia: or
hospitalized COVID pneumonia with hypoxia.

In one embodiment, administration of the IL-6 antagonist treats:
1. coronavirus infection;
2. COVID-19 infection;
3. inflammation associated with coronavirus;
4. cytokine release associated with viral infection;
5. cytokine release associated with coronavirus.

According to certain embodiments, the IL-6 antagonist:
binds IL-6 receptor;
binds IL-6;
is tocilizumab, satralizumab, sarilumab, NI-120, vobarilizumab, sirukumab, olokizumab, clazakizumab, siltuximab, EBI-031, or olamkicept;
is sarilumab;
is preferably tocilizumab.

According to certain embodiments, the IL-6 antagonist is tocilizumab and is administered as a first weight-based 8 mg/kg intravenous dose of tocilizumab (e.g. wherein the first dose is ≤800 mg of tocilizumab) optionally followed by a second weight-based 8 mg/kg intravenous dose of tocilizumab 8-24 hours after the first dose (e.g. wherein the second weight-based dose of tocilizumab is ≤800 mg).

According to certain embodiments, the IL-6 antagonist is combined with at least one further agent (e.g. one, two, three, four, or more further agents) to treat the patient, e.g. where the further agent comprises:
anti-viral (e.g. remdesiver, lopinavir/ritonavir, chloroquine phosphate, hydroxychloroquine, umifenovir and/or favipiravir), optionally combined with α-interferon, ribavirin, and/or azithromycin;
corticosteroid (e.g. prednisone, prednisolone, methylprednisolone, methylprednisolone sodium succinate, dexamethasone, dexamethasone triamcinolone, hydrocortisone, and/or betamethasone);
another anti-inflammatory drug (e.g. interferon gamma antagonist, interleukin 1 antagonist, another IL-6 antagonist, complement factor 5a antagonist, steroid, anti-ST2, IL-22 Fc, and/or statin);
another immunomodulator (e.g. another IL-6 antagonist, sarilumab, anakinra, baricitinib, canakinumab, and/or ruxolitinib);
anti-coagulant (e.g. heparin);
anti-fibrotic or tyrosine kinase inhibitor (e.g. imatinib) or pirfenidone;
anti-viral antibody or cocktails thereof (e.g. REGN-COV2);
antibodies (e.g. convalescent plasma, hyperimmune immunoglobulins, convalescent plasma-derived hyperimmune globulin, monoclonal antibody targeting SARS-CoV-2): and/or
SARS-CoV-2 vaccine.

In one embodiment, the IL-6 antagonist is administered to the patient in combination with remdesivir, e.g. as an initial one-time dose of 200 mg followed by 100 mg per day, for 5 to 10 total doses

In one embodiment, only a single weight-based dose, 8 mg/kg (5800 mg) of tocilizumab is administered to the patient.

In one embodiment, only two weight-based doses, each being 8 mg/kg (each ≤800 mg), of tocilizumab are administered to the patient.

In one embodiment, a second dose of tocilizumab is administered, for example:
after the patient experiences no improvement or worsening of clinical status after the first dose;
after the patient experiences no improvement or ≥one-category worsening on an ordinal scale of clinical status following the first dose;
after the patient experiences ? one-category worsening on an ordinal scale of clinical status (e.g. a 7-category ordinal scale) following the first dose.

In one embodiment, treatment with the IL-6 antagonist (e.g. tocilizumab) achieves a greater improvement in clinical outcome than standard of care (SOC).

In one embodiment, treatment with the IL-6 antagonist is associated with acceptable safety outcome compared with standard of care (SOC), with exemplary safety outcomes include any one or more of:
incidence and severity of adverse events;
severity of adverse events determined according to National Cancer Institute Common Terminology Criteria for Adverse Events (NCI CTCAE) v5.0;
COVID-19 (SARS-CoV-2) viral load over time;
time to reverse-transcriptase polymerase chain reaction (RT-PCR) virus negativity;
post-treatment infection; and
change from baseline in targeted clinical laboratory test results.

Herein, SOC for pneumonia, in particular viral pneumonia (such as COVID-19 pneumonia) includes any one or more of (e.g. one, two, or three of):
1. supportive care;
2. one or more anti-viral agent(s);
3. one or more corticosteroid(s), e.g. low dose corticosteroid(s).

In one embodiment, the IL-6 antagonist is combined with supportive care. Example of supportive care, include, without limitation:
1. oxygen therapy (e.g. via face mask or nasal cannula: high-flow nasal oxygen therapy or non-invasive mechanical ventilation; invasive mechanical ventilation; lung expansion via extracorporeal membrane oxygenation (ECMO), etc.);
2. circulation support (e.g. fluid resuscitation, boost microcirculation, and/or vasoactive drugs);
3. renal replacement therapy;
4. plasma therapy;
5. blood purification therapy;
6. Xuebijing Injection (e.g. 100 mL/day twice a day);
7. microecological agents (e.g. probiotics, prebiotics, and synbiotics); and/or
8. antibodies (e.g., convalescent plasma, hyperimmune immunoglobulins, convalescent plasma-derived hyperimmune globulin, monoclonal antibodies targeting COVID-19), etc.

In one embodiment, IL-6 antagonist is combined with more anti-viral agents (preferably only one or two) anti-viral agent(s). Exemplary anti-viral treatments include, without limitation:
1. remdesiver (e.g. RDV is administered 200 mg on Day 1 followed by RDV 100 mg on Days 2, 3, 4, and 5 or RDV 200 mg on Day 1 followed by RDV 100 mg on Days 2, 3, 4, 5, 6, 7, 8, 9, and 10).
2. alpha-interferon (e.g. via nebulization; e.g. about 5 million units or equivalent per time for adult, add 2 mL of sterile water for injection; e.g. via aerosol inhalation twice per day);
3. lopinavir/ritonavir (e.g. 200 mg/50 mg per capsule, 2 capsules each time, twice per day for adults, e.g. ≤10 days);
4. ribavirin (e.g. combined with alpha-interferon or lopinavir/ritonavir, e.g. 500 mg for adults per time, 2-3 times per day intravenously, e.g. ≤10 days);
5. Chloroquine phosphate or hydroxychloroquine (e.g. for adults from 18 to 65 years of age; e.g. if the body weight is greater than 50 kg, 500 mg per time, twice per day for 7 days; if the body weight is less than 50 kg, 500 mg per time, twice per day for day 1 and day 2; 500 mg per time, once per day for day 3 to 7), optionally together with azithromycin;
6. Umifenovir (e.g. 200 mg for adults, e.g. three times per day, e.g. ≤10 days), and
7. Favipiravir (e.g. 1600 mg twice daily on day 1, then 600 mg twice daily thereafter for 7-10 or 14 days).

In one embodiment, the IL-6 antagonist is combined with corticosteroid(s), e.g.
1. prednisone, prednisolone, methylprednisolone, methylprednisolone sodium succinate, dexamethasone, dexamethasone triamcinolone, hydrocortisone, and/or betamethasone;
2. “low dose” corticosteroid;
3. corticosteroid (e.g. ≤1-2 mg/kg/day);
4. methylprednisolone (e.g. ≤1-2 mg/kg/day);
5. methylprednisolone (e.g. ≤1-2 mg/kg/day for 3-5 days);
6. dexamethasone (e.g. oral or iv 6 mg once daily for up to 10 days).

These additional drugs as set forth herein are generally used in the same dosages and with administration routes as used hereinbefore or about from 1 to 99% of the heretofore-employed dosages. If such additional drugs are used at all, preferably, they are used in lower amounts than if the first medicament were not present, especially in subsequent dosings beyond the initial dosing with the first medicament, so as to eliminate or reduce side effects caused thereby.

The combined administration of an additional drug includes co-administration (concurrent administration), using separate formulations or a single pharmaceutical formulation, and consecutive administration in either order, wherein preferably there is a time period while both (or all) active agents (medicaments) simultaneously exert their biological activities.

Further details of the invention are illustrated by the following non-limiting Example. The disclosures of all citations in the specification are expressly incorporated herein by reference.

### Example 1

### Results of Empacta: A Randomized, Double-Blind, Placebo-Controlled, Multicenter Study to Evaluate the Efficacy and Safety of Tocilizumab in Hospitalized Patients with Covid-19 Pneumonia

### Patient Selection

### Inclusion Criteria:

18+ years with confirmed SARS CoV 2 (COVID 19) infection by a positive PCR of any specimen (e.g., respiratory, blood, urine, stool, other bodily fluid) and evidenced by CT scan or chest X-ray)
SpO2<94% while on ambient air

### Exclusion Criteria:

Require continuous positive airway pressure (CPAP), bilevel positive airway pressure (BIPAP), or invasive mechanical ventilation
In the opinion of the investigator, progression to death is imminent and inevitable within the next 24 hours, irrespective of the provision of treatments
Suspected active bacterial, fungal, viral, or other infection (besides COVID-19)
At screening. ALT or AST >5×ULN: ANC <1000/mL; Platelet count <50,000/Ml

### Efficacy Endpoints

Primary and Secondary Efficacy Endpoints are summarized below. Note that all efficacy analyses were stratified by age (≤60, >60 years). Efficacy endpoints were tested in a hierarchical manner, starting with the primary efficacy endpoint and gated.

### Primary Efficacy Endpoint:

Cumulative proportion of patients with death or requiring mechanical ventilation by Day 28 (time to event analysis)

### Secondary Efficacy Endpoints:

1. Time to hospital discharge or “ready for discharge” up to Day 28 (defined as time to hospital discharge or “ready for discharge” up to Day 28 on the 7-category ordinal scale).
2. Time to improvement in ordinal clinical status up to Day 28 (defined as time to at least a 2-category, or 1-category if baseline ordinal scale is 2, improvement in the 7-category ordinal scale up to Day 28).
3. Time to clinical failure up to Day 28 (defined as the time to death, mechanical ventilation, ICU admission, or 2-category worsening in the 7-category ordinal scale if patient was already admitted to the ICU at baseline, or withdrawal (whichever occurs first)).
4. Mortality by Day 28.

### Results

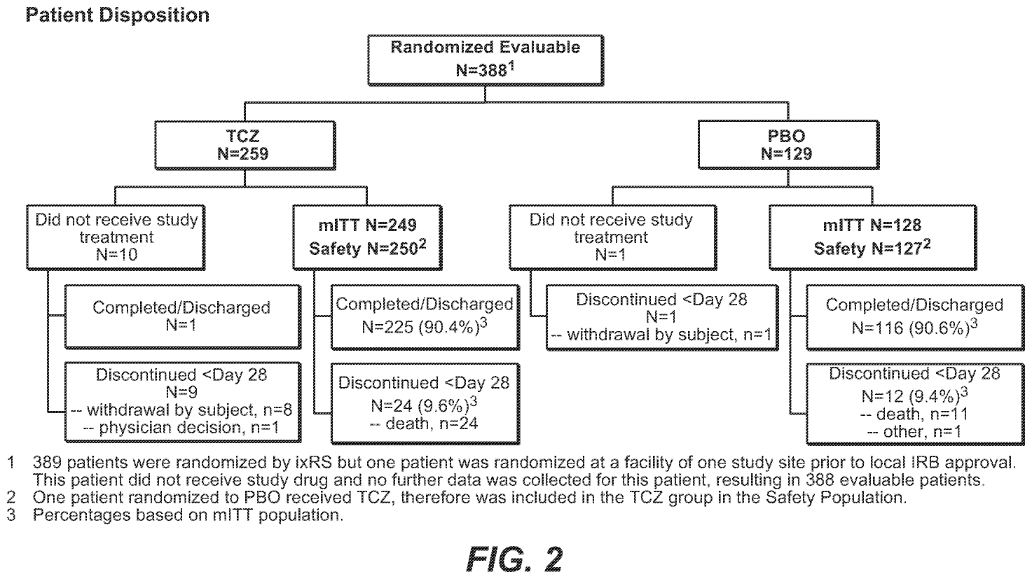

The results of the EMPACTA clinical trial are shown in FIGS. 2-14.

FIG. 2 depicts patient disposition. 389 patients were enrolled (388 evaluable) from US, Mexico, Kenya, South Africa, Peru, and Brazil.

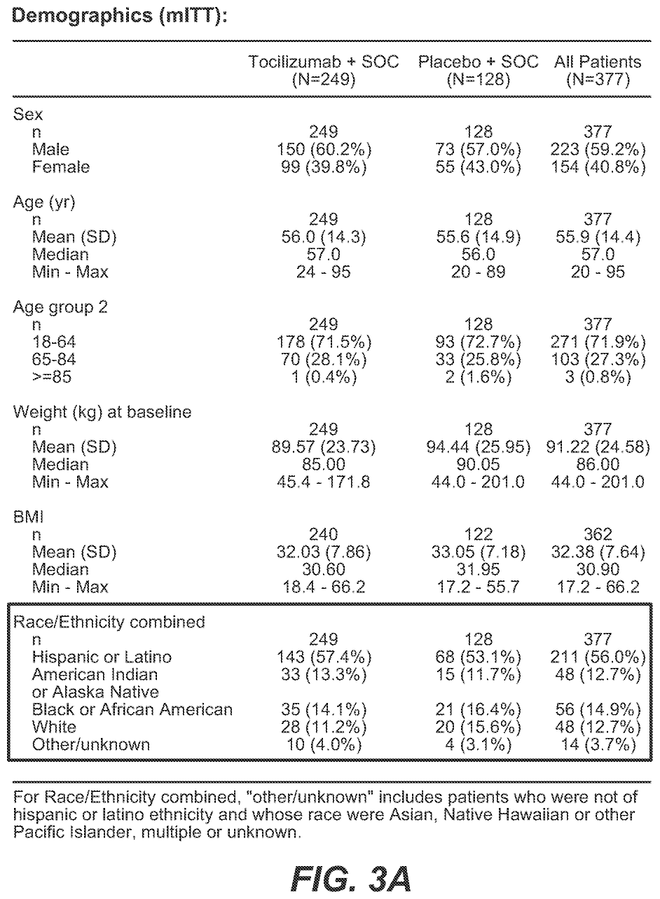

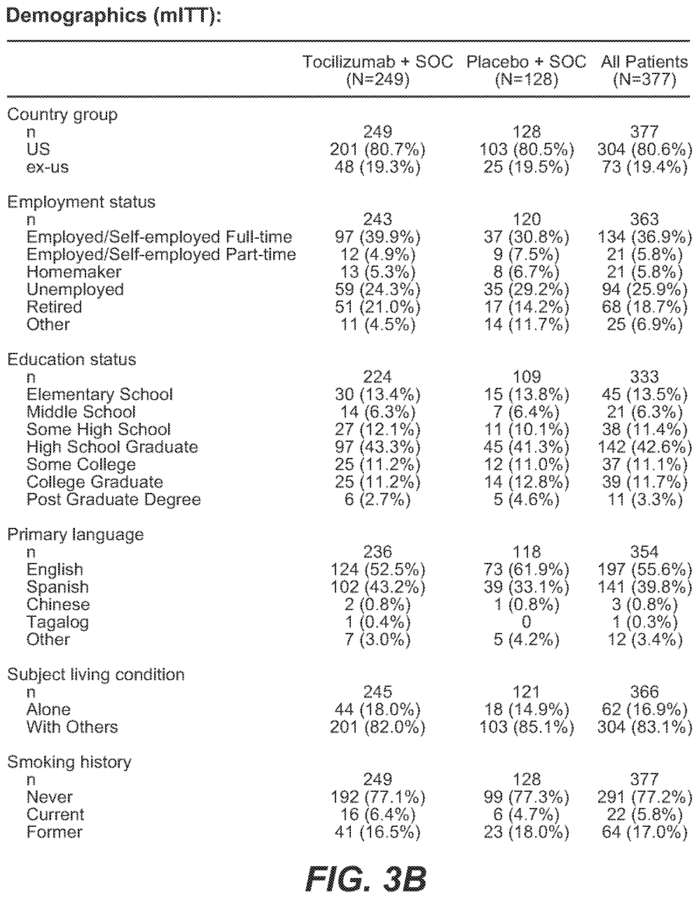

The patient demographics as shown in FIGS. 3A-B demonstrate that:
Groups were generally well balanced.
Approximately 60% males.
Mean age was 56 years, 72% were <65 years of age.
Mean weight was 91 kg, mean body mass index (BMI) was 32
87% minority patients: Hispanic/Latino 56%, Black/African American 15%, American Indian/Alaska Native/Other 16%.
Approximately 81% US patients
Employment status, education status, and smoking history were similar between the groups.

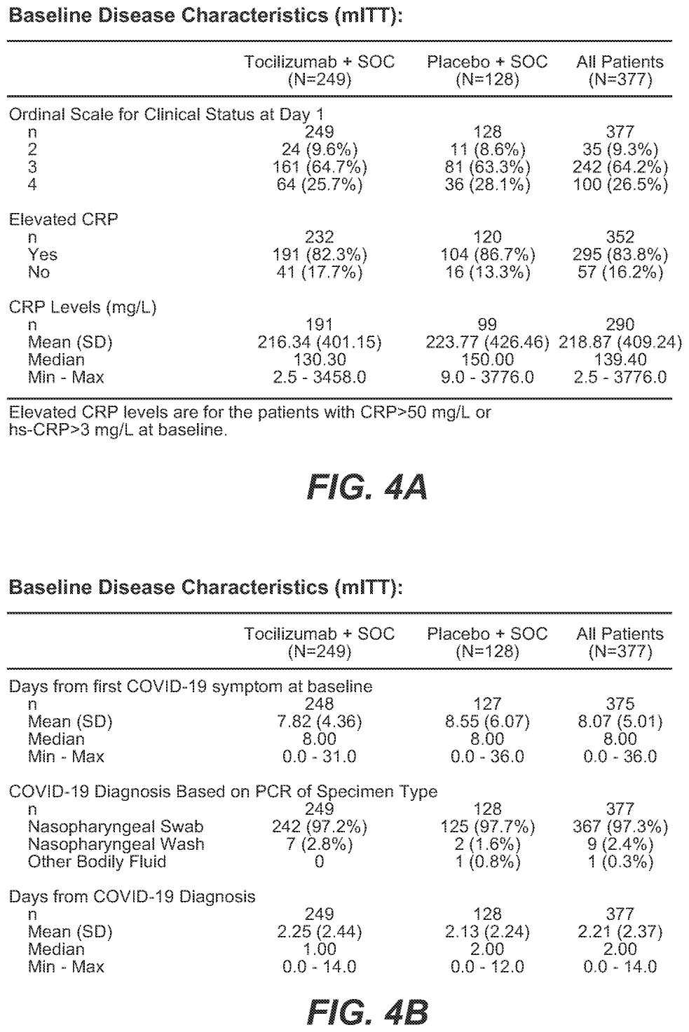

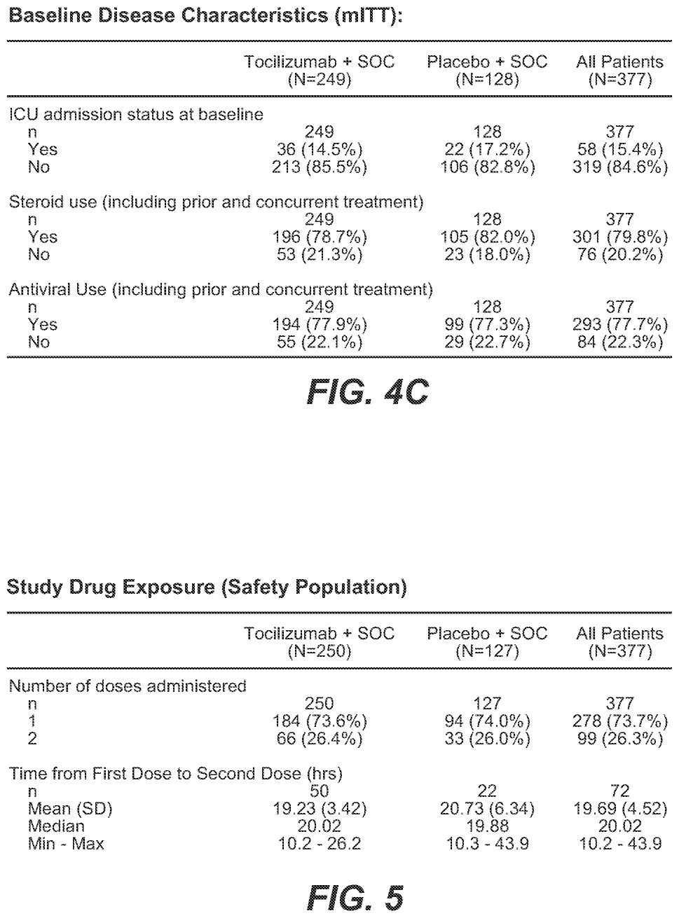

Baseline disease characteristics as depicted in FIGS. 4A-C showed that:
Groups were balanced with respect to baseline ordinal scale and inflammatory markers.
Overall 84% of patients had elevated CRP at baseline.
Mean time from first COVID symptoms to baseline was 8 days in both groups.
Mean time from COVID diagnosis to baseline was 2 days.
Steroid use (80%) and antiviral use (78%) were similar between groups.

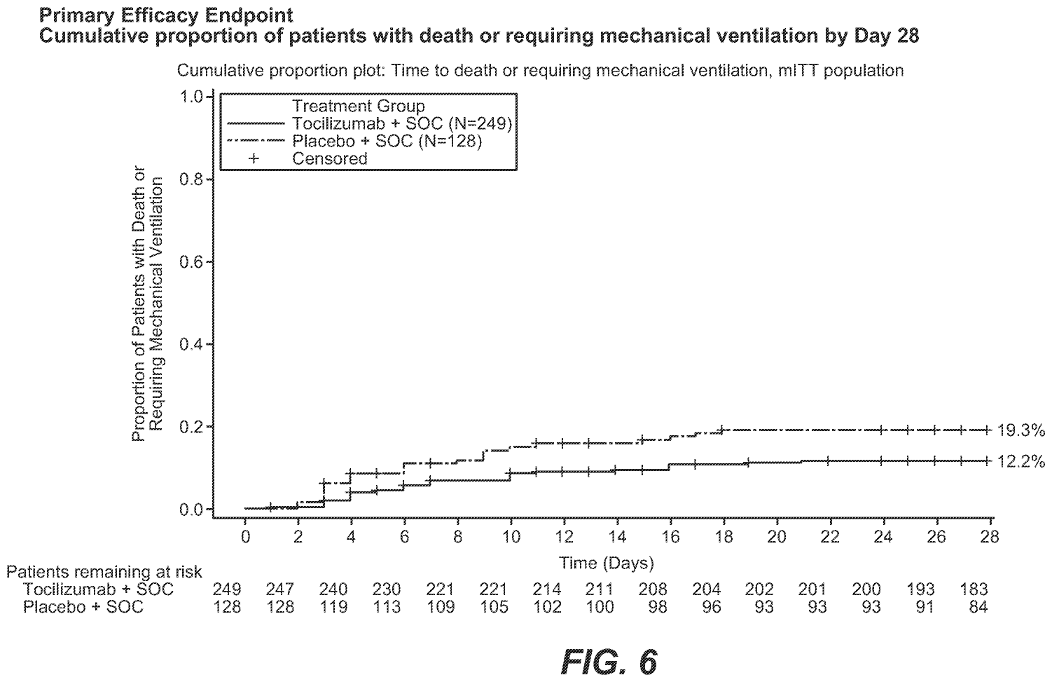

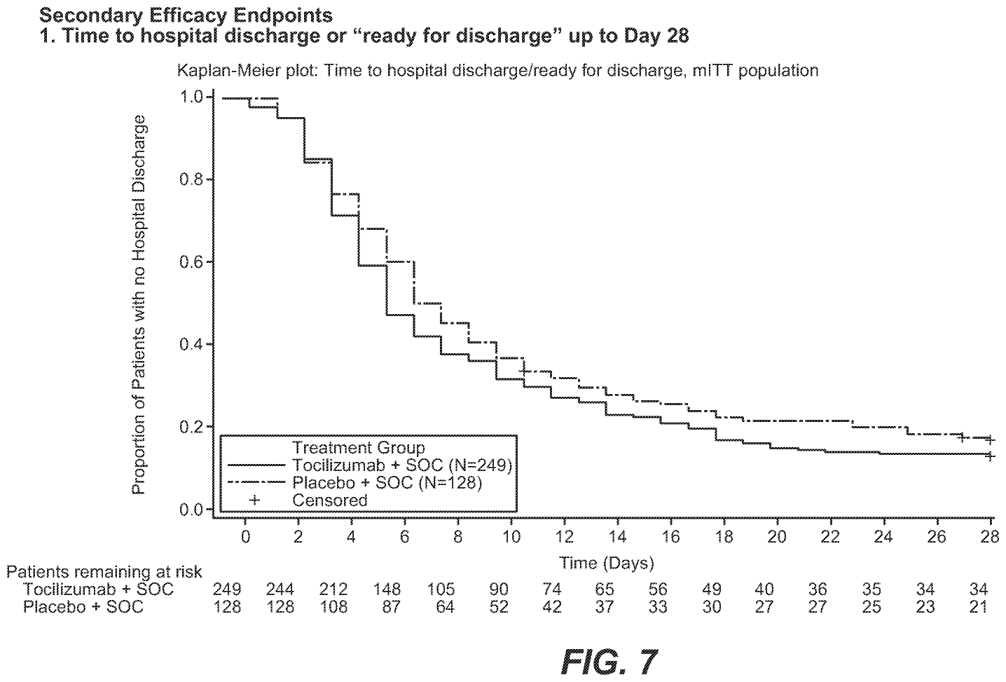

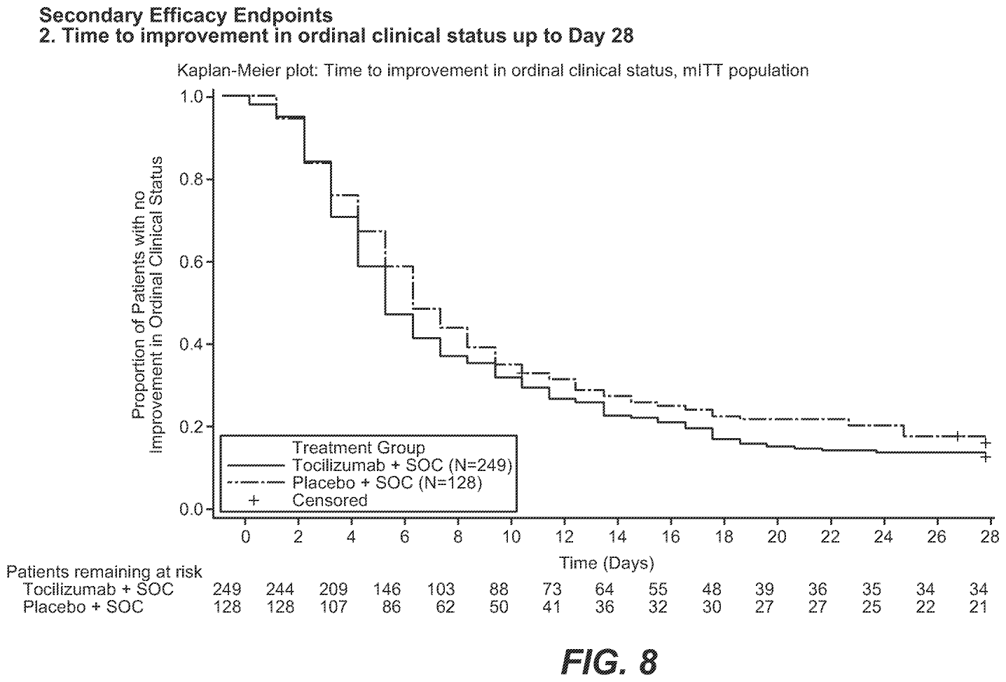

The Primary Efficacy Endpoint was cumulative proportion of patients with death or requiring mechanical ventilation by Day 28 and the results of the endpoint are depicted in FIG. 6. As shown in this figure, the primary endpoint was statistically significant:
Day 28 cumulative event rate (95% CI) was 12.2% (8.61%, 17.04%) in TCZ+SOC compared to 19.3% (13.38%, 27.45%) in PBO+SOC (log-rank test p-value=0.0348)
Hazard ratio (95% CI) [ref=PBO]: 0.56 (0.32, 0.97)—TCZ+SOC significantly reduced the risk of death or requiring mechanical ventilation by 44% when compared to PBO+SOC.

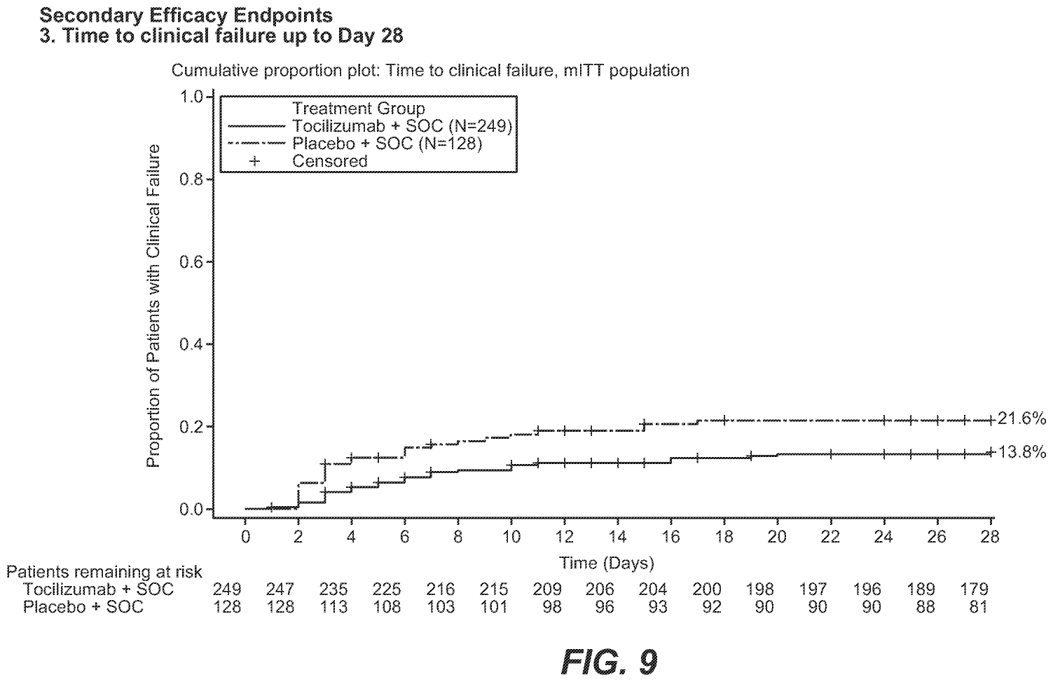

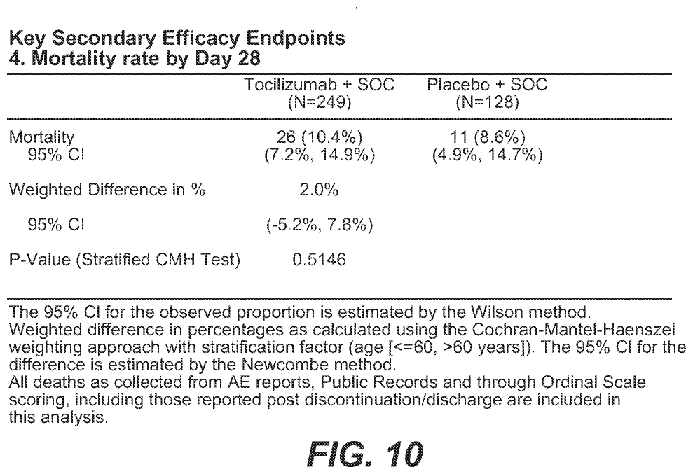

Secondary Efficacy Endpoints are depicted in FIGS. 7-10. As shown in these figures:
FIG. 7: time to hospital discharge or “ready for discharge” up to Day 28, no statistical significance found:
Median time (95% CI) to hospital discharge was 6 days (6.0, 7.0) in TCZ+SOC compared to 7.5 days (7.0, 9.0) in PBO+SOC (log-rank test p-value=0.2456)
Hazard ratio (95% CI) [ref=PBO]: 1.16 (0.90, 1.48)
FIG. 8: time to improvement in ordinal clinical status up to Day 28, no statistical significance found:
Median time (95% CI) to clinical improvement was 6 days (6.0, 7.0) in TCZ+SOC compared to 7 days (6.0, 9.0) in PBO+SOC (log-rank test p-value=0.2597)
Hazard ratio (95% CI) [ref=PBO]: 1.15 (0.90, 1.47)
FIG. 9: time to clinical failure up to Day 28, endpoint met nominal statistical significance:
Median time to event was not reached in both groups (log-rank test p value=0.0217)
Hazard ratio (95% CI) [ref=PBO]: 0.55 (0.33, 0.92)
FIG. 10: mortality rate by Day 28:
mortality rates were comparable between groups:
2 deaths in TCZ group were reported within 28 days during safety follow-up after hospital discharge.

### Summary of Efficacy Analysis Results

Primary

| Cumulative proportion of patients | Cumulative proportion of event: |
|---|---|
| who died or required mechanical | TCZ = 12.2%; PBO = 19.3% |
| ventilation by Day 28 (time to | Log-rank test p-value = 0.0348 |
| event analysis): was significant | Hazard ratio [95% CI] = 0.56 |
| | [0.32, 0.97] |

Key Secondary

| | | |
|---|---|---|
| 1 | Time to hospital dis- | Median time to event (days): TCZ = 6; |
| | charge or “ready for | PBO = 7.5 |
| | discharge” to Day 28: | Log-rank test p-value = 0.2456 |
| | was not significant | Hazard ratio [95% CI] = 1.16 [0.90, |
| | | 1.48] |
| 2 | Time to improvement in | Median time to event (days): TCZ = 6; |
| | ordinal clinical status | PBO = 7 |
| | to Day 28 | Log-rank test p-value = 0.2597 |
| | | Hazard ratio [95% CI] = 1.15 [0.90, |
| | | 1.47] |
| 3 | Time to clinical failure | Median time to event (days): TCZ = NE; |
| | up to Day 28 | PBO = NE |
| | | Cumulative proportion of event: TCZ = |
| | | 13.8%; PBO = 21.6% |
| | | Log-rank test p-value = 0.0217 |
| | | Hazard ratio [95% CI] = 0.55 [0.33, |
| | | 0.92] |
| 4 | Difference in mortality | Mortality rate: TCZ = 10.4%; PBO = |
| | rate by Day 28 | 8.6% |
| | | Cochran-Mantel-Haenszel test p-value = |
| | | 0.5146 |
| | | Difference in mortality rates [95% CI]: |
| | | 2.0% [−5.2%, 7.8%] |

Note:
Testing performed in a hierarchical manner to control the overall study-wide Type 1 error rate at the 5% significance level.
P-values were based on tests stratified by age.
NE = not estimable.

### Safety Data

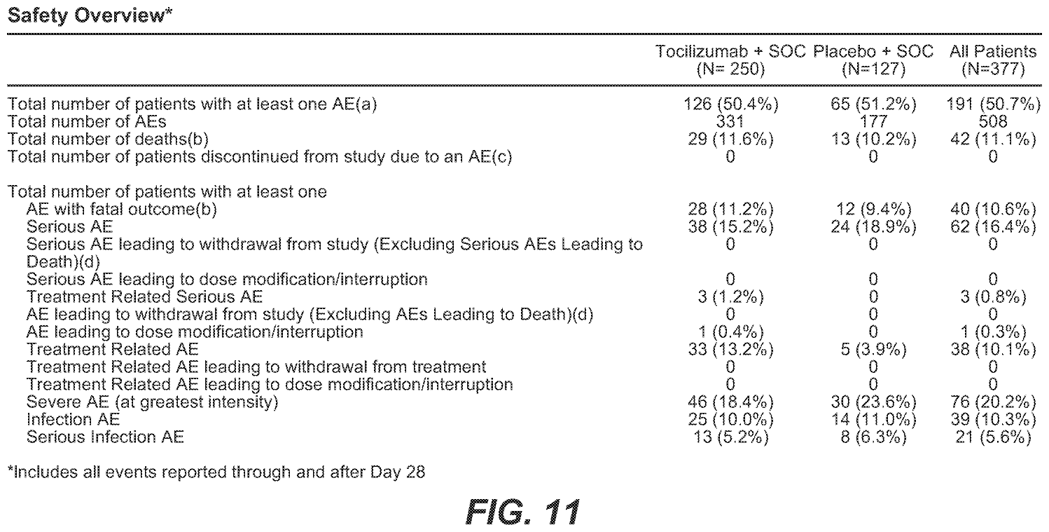

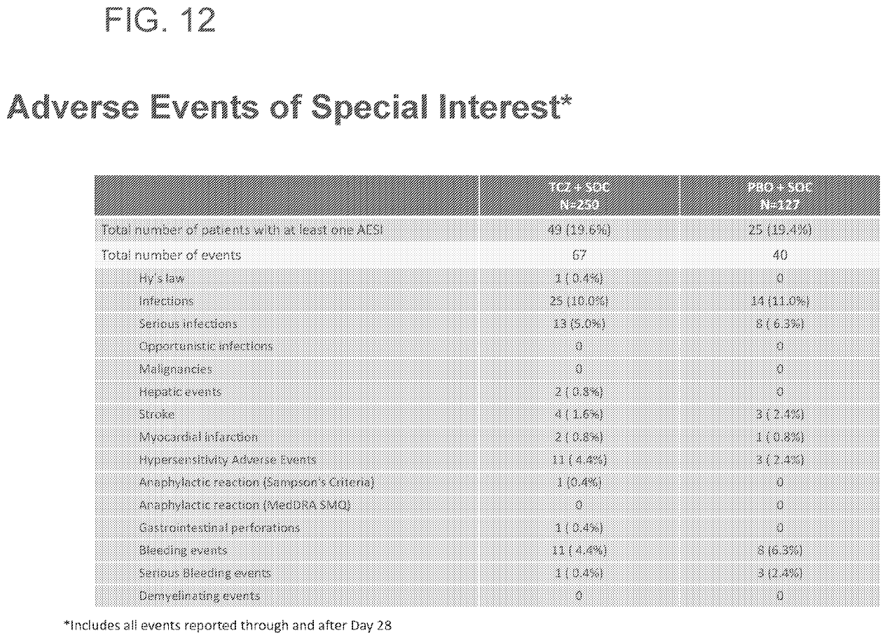

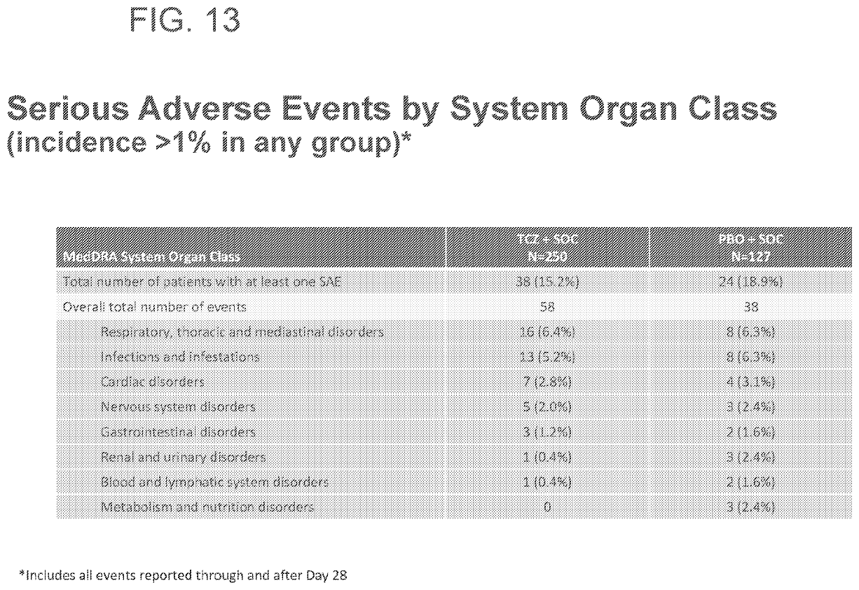

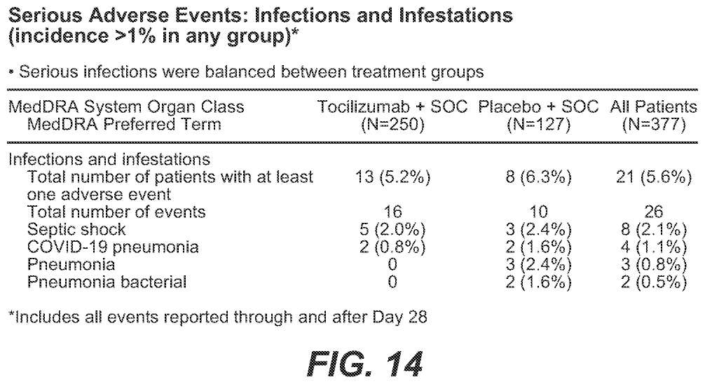

Safety data are depicted in FIGS. 11-14 which show:
FIG. 11: safety overview:
   SAEs, Serious infections and fatal AEs were balanced between groups
   No new safety signals
FIG. 12: adverse events of special interest (AESIs):
   AESIs were balanced between groups and comparable with known profile of TCZ
FIG. 13: serious adverse events (SAE) by system organ class (incidence >1% in any group):
   SAEs were balanced between treatment groups
FIG. 14: serious adverse events: infections and infestations (incidence >1% in any group)
   Serious infections were balanced between treatment groups.

### CONCLUSIONS

### Efficacy

TCZ+SOC significantly reduced the risk of death or requiring mechanical ventilation when compared to PBO+SOC.
Time to clinical failure was the only key secondary efficacy endpoint with nominal significance.

### Safety

No new safety signals identified.
Overall AE profiles balanced between TCZ and PBO, including infections and serious infections.

### Overall Study Conclusion

EMPACTA demonstrated efficacy and safety of TCZ+SOC over PBO+SOC in reducing the risk of death or requiring mechanical ventilation in hospitalized COVID-19 pneumonia patients who did not require mechanical ventilation at baseline. Overall, mortality rates between the treatment groups were not different at Day 28.

What is claimed is:

1. A method of treating pneumonia in a patient who is not mechanically ventilated comprising administering an IL-6 antagonist to the patient in an amount effective to prevent the patient from dying or receiving mechanical ventilation.

2. The method of any claim 1, wherein the pneumonia is viral pneumonia.

3. The method of any one of claim 1 or claim 2, wherein the pneumonia is moderate, severe, or critical pneumonia.

4. The method of claim 3, wherein the pneumonia is severe pneumonia.

5. The method of any one of the preceding claims, wherein the pneumonia is coronavirus pneumonia.

6. The method of claim 5, wherein the pneumonia is COVID-19 pneumonia, Middle East respiratory syndrome (MERS-CoV) pneumonia, or severe acute respiratory syndrome (SARS-CoV) pneumonia.

7. The method of claim 6, wherein the pneumonia is COVID-19 pneumonia.

8. The method of any one of the preceding claims, wherein the patient is hospitalized.

9. The method of any one of the preceding claims, wherein the patient has hypoxia.

10. The method of any one of the preceding claims, wherein administration of the IL-6 antagonist prevents the patient from dying or receiving mechanical ventilation within about 28 days from a first administration of the IL-6 antagonist.

11. The method of claim 10, wherein the estimated cumulative proportion of patients with death or mechanical ventilation at Day 28 is about 12% with IL-6 antagonist administration compared with about 19% in patients not receiving the anti-IL-6 antagonist.

12. The method of any one of the preceding claims, wherein the patient further receives standard of care.

13. The method of claim 12, wherein administration of the IL-6 antagonist reduces the risk of the patient dying or receiving mechanical ventilation compared to the patient not receiving the IL-6 antagonist but receiving the standard of care (SOC).

14. The method of claim 13, wherein administration of the IL-6 antagonist reduces the risk of the patient dying or receiving mechanical ventilation by at least about 40% compared with the patient not receiving the IL-6 antagonist but receiving the SOC.

15. The method of any one of claims 12 to 14, wherein the SOC comprises administration of anti-viral (e.g. remdesiver or azithromycin) and/or administration of corticosteroid (e.g. dexamethasone or prednisone).

16. The method of any one of the preceding claims, wherein administration of the IL-6 antagonist further reduces likelihood of clinical failure.

17. The method of claim 16, wherein clinical failure comprises time to: death, mechanical ventilation, ICU admission, and/or 2-category worsening in the 7-category ordinal scale wherein the patient was already admitted to the ICU prior to treatment.

18. The method of claim 17, which reduces the risk of clinical failure up to Day 28.

19. The method of any one of the preceding claims, wherein the IL-6 antagonist binds IL-6 receptor.

20. The method of claim 19, wherein the IL-6 antagonist is tocilizumab.

21. The method of claim 20, wherein the effective amount of tocilizumab comprises a first weight-based 8 mg/kg intravenous dose of tocilizumab optionally followed by a second weight-based 8 mg/kg intravenous dose of tocilizumab 8-24 hours after the first dose.

22. The method of any one of the preceding claims, further comprising administering at least one further agent to treat the patient, wherein the further agent comprises:
a. anti-viral (e.g. remdesiver, azithromycin, lopinavir/ritonavir, chloroquine phosphate, hydroxychloroquine, umifenovir and/or favipiravir), optionally combined with α-interferon, ribavirin, and/or azithromycin; and/or
b. corticosteroid (e.g. prednisone, prednisolone, methylprednisolone, methylprednisolone sodium succinate, dexamethasone, dexamethasone triamcinolone, hydrocortisone, and/or betamethasone).

23. The method of claim 22, wherein the further agent comprises an anti-viral, and the anti-viral comprises remdesivir or azithromycin.

24. The method of claim 22, wherein the further agent comprises a corticosteroid, and the corticosteroid comprise dexamethasone or prednisone.

25. A method of treating viral pneumonia in a patient comprising who is not mechanically ventilated comprising administering a combination of an IL-6 antagonist and remdesivir to the patient in an amount effective to prevent the patient from dying or receiving mechanical ventilation.

26. The method of claim 25, wherein the IL-6 antagonist is tocilizumab.

27. The method of claim 26, wherein the effective amount of tocilizumab comprises a first weight-based 8 mg/kg intravenous dose of tocilizumab optionally followed by a second weight-based 8 mg/kg intravenous dose of tocilizumab 8-24 hours after the first dose.

28. The method of any one of claims 25 to 27, wherein the effective amount of remdesivir comprises an initial one-time dose of 200 mg followed by 100 mg per day, and wherein 5 to 10 total doses of remdesivir are administered to the patient.

29. A method of treating viral pneumonia in a patient who is not mechanically ventilated comprising administering a combination of an IL-6 antagonist and a corticosteroid to the patient in an amount effective to prevent the patient from dying or receiving mechanical ventilation.

30. The method of claim 29, wherein the IL-6 antagonist is tocilizumab.

31. The method of claim 30, wherein the effective amount of tocilizumab comprises a first weight-based 8 mg/kg intravenous dose of tocilizumab optionally followed by a second weight-based 8 mg/kg intravenous dose of tocilizumab 8-24 hours after the first dose.

32. The method of any one of claims 29 to 31, wherein the corticosteroid comprises dexamethasone.

33. The method of claim 32, wherein the dexamethasone is administered orally or intravenously 6 mg once daily for up to 10 days.


SEQUENCE LISTING (8 sequences)

SEQ ID NO: 1 - protein, 214 aa, Artificial Sequence [sequence omitted]

SEQ ID NO: 2 - protein, 448 aa, Artificial Sequence
  Tocilizumab heavy chain
  MOD_RES (1)..(1): Pyroglutamic Acid
XVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 10 aa, Artificial Sequence
  Tocilizumab CDR L1
RASQDISYLN

SEQ ID NO: 4 - protein, 7 aa, Artificial Sequence
  Tocilizumab CDR L2
YTSRLHS

SEQ ID NO: 5 - protein, 8 aa, Artificial Sequence
  Tocilizumab CDR L3
QGNTLPYT

SEQ ID NO: 6 - protein, 6 aa, Artificial Sequence
  Tocilizumab CDR H1
SDHAWS

SEQ ID NO: 7 - protein, 15 aa, Artificial Sequence
  Tocilizumab CDR H2
YISYSGITYNPSLKS

SEQ ID NO: 8 - protein, 9 aa, Artificial Sequence
  Tocilizumab CDR H3
SLARTAMDY